(19) United States
(12) Patent Application Publication
(10) Pub. No.: US 2026/0028305 A1
(43) Pub. Date: Jan. 29, 2026

(54) PROCESS FOR CONTINUOUS CATALYTIC HYDROGENATION OF MDA

(71) Applicant: Evonik Operations GmbH, Essen (DE)

(72) Inventors: Hanns Kuhlmann, Sprockhoevel (DE); Armin Matthias Rix, Marl (DE); Niklas Paul, Marl (DE); Lea Wessner, Dortmund (DE); Maria Vargas Gómez, Haltern am See (DE); Tobias Winkler, Duelmen (DE); Florian Boeck, Witten (DE); Daniel Sudhoff, Bergkamen (DE); Christian Lettmann, Coesfeld (DE)

(73) Assignee: Evonik Operations GmbH, Essen (DE)

(21) Appl. No.: 19/277,543

(22) Filed: Jul. 23, 2025

(30) Foreign Application Priority Data
Jul. 26, 2024 (EP) 24191102.3

(51) Int. Cl. C07C 209/72; C07C 209/86

(52) U.S. Cl. CPC C07C 209/72; C07C 209/86; C07C 2601/14

(57) ABSTRACT

A plant for hydrogenation of methylenedianiline with a hydrogen donor has a conditioning unit for the reactants, a reactor unit and a separation unit. The reactor unit has at least one fixed bed reactor as main reactor with an immobile catalyst packing, and the separation unit has at least a first separation stage having at least one apparatus for removing the solvent, and wherein a second separation stage has at least one apparatus for separation of at least one reactant and/or at least one by-product from the PACM product.

### CROSS-REFERENCE TO RELATED APPLICATIONS

This patent application claims priority to European Patent Application No. 24191102.3, filed on Jul. 26, 2024, in the European Patent Office, the entire disclosure of which is hereby incorporated by reference herein.

### BACKGROUND OF THE INVENTION

### Field of the Invention

The invention relates to a plant and to a process for continuous catalytic hydrogenation of MDA, especially for production of methylenebis(cyclohexylamine), such as 4,4′-diaminodicyclohexylmethane (PACM) in particular.

### Description of Related Art

Processes for hydrogenation of organic compounds, especially for hydrogenation of aromatic compounds to the corresponding cyclohexane derivatives, are already known from the prior art.

Methylenebis(cyclohexylamine) is a cycloaliphatic amine that is solid or liquid under standard conditions (SATP) and is typically produced by liquid phase hydrogenation of MDA. The acronym MDA was introduced historically as an abbreviation for the product mixture which is formed in the reaction of aniline and formaldehyde and comprises mainly “methylenedianiline” (diaminodiphenylmethane), and is still used to describe the process product, which is now produced on an industrial scale. The hydrogenation product, comprising mainly methylenebis(cyclohexylamine), is therefore often also referred to as H12MDA.

Because of the process for production thereof, MDA is typically a mixture of different diaminodiphenylmethanes. It consists mainly of 4,4′-diaminodiphenylmethane. However, 2,4′ and 2,2′ isomers may also be present. MDA may also contain reaction products having three or more aromatic rings that are formed in the reaction of aniline and formaldehyde, in particular those having three or more phenyl rings. These reaction products having three or more aromatic rings are also referred to as polynuclear compounds.

Because of the high proportion of 4,4′-diaminodiphenylmethane in the MDA used, commercially available methylenebis(cyclohexylamine) is mostly 4,4′-diaminodicyclohexylmethane or bis(para-aminocyclohexyl) methane. Because of the potential presence of the corresponding 2,4′- and 2,2′-diaminophenylmethane isomers in the MDA, 2,4′-diaminodicyclohexylmethane and 2,2′-diaminodicyclohexylmethane may also be present in methylenebis(cyclohexylamine). In addition, hydrogenated MDA may also contain (optionally partly) hydrogenated polynuclear compounds as well as methylenebis(cyclohexylamine).

U.S. Pat. No. 5,578,546 A discloses that a process for producing methylenebis(cyclohexylamine) was first described in 1947 and converted to industrial scale in 1965. Hydrogenation of MDA is highly exothermic. For example, WO 2010/069484 A1 indicates an enthalpy of reaction of −1600 kJ/mol.

As a result of hydrogenation, various diastereoisomers are formed depending on the process. The 4,4′-diaminodicyclohexylmethane (PACM) product derived from 4,4′-diaminodiphenylmethane may take the form of trans/trans, cis/cis and cis/trans isomers and is therefore generally a mixture of these isomers with different proportions. The melting point of the compound rises with rising trans/trans content. Therefore, the fields of application vary significantly depending on the isomer content: While methylenebis(cyclohexylamine) qualities with a low trans/trans content (e.g. 10-30% by weight) are used in the field of amine and isocyanate crosslinkers, especially in the field of two-component resins, qualities with a high trans/trans content (e.g. >48% by weight) are mainly used as regulator in polyamide compounds. The production of products with a low trans/trans content in particular is a challenge, since the thermodynamic equilibrium, as described in U.S. Pat. No. 3,636,108 A, is in the range of significantly higher trans/trans contents (up to 51.2%). In addition, U.S. Pat. No. 2,606,925 A shows that the equilibrium can be shifted retrospectively by prolonged heat treatment in the direction of a higher proportion of trans/trans isomer.

The composition of the hydrogenation product also depends on the composition of the MDA used: MDA is often used in qualities from MDA50 to MDA100, with the number between 50 and 100 indicating the content of diaminodiphenylmethanes in the MDA mixture. MDA50 is an MDA quality which, as explained above, for process-related reasons, contains about 50% by weight of diaminodiphenylmethanes and 50% by weight of polynuclear compounds. The individual polynuclear compounds can be referred to as trinuclear compounds, tetranuclear compounds, etc. according to the number of aromatic nuclei present. MDA50 is the quality produced in the highest volume and is mainly processed to methylene dicyclohexyl diisocyanate (MDI). MDA100 is pure MDA or diaminodiphenylmethane without polynuclear compounds. MDA85 or MDA90 are other grades of medium purity available on the market. When patent specifications relating to the process for production of methylenebis(cyclohexylamine) address the purity of the MDA quality, the quality in question is usually MDA100 (e.g. CN 110204447 B). US 2005/261525 A1, by contrast, prefers the hydrogenation of MDA50. The hydrogenated oligomeric amines obtained here as high boilers are suitable as crosslinkers having particularly low vapour pressures for a range of specialty applications, as shown by US 2004/162409 A1.

The content of (possibly partly) hydrogenated polynuclear compounds in the product decreases in the order of the reactants MDA50, MDA85, MDA90, MDA100, since the content of polynuclear compounds decreases from MDA50 to MDA100.

WO 2009/153123 A1 discloses a continuous process and a reactor for hydrogenation of organic compounds in a polyphasic multistage system in the presence of a homogeneous or heterogeneous catalyst. Catalysts proposed include precious metals such as platinum, palladium, ruthenium and rhodium, or other transition metals such as molybdenum, tungsten and chromium. It is possible here for the heterogeneous catalysts to be disposed on support materials, for example carbon, aluminium oxide, silicon dioxide, zirconium dioxide, zeolites, aluminosilicates or mixtures of these support materials. Substrates used in this process are preferably aromatic compounds containing amino substituents, for example MDA, polymer MDA, aniline, 2,4-diaminotoluene, 2,6-diaminotoluene, o-phenylenediamine, etc. The heterogeneous catalysts are used in suspension.

DE 19533718 A1 discloses a process for hydrogenation of aromatic compounds in which at least one amino group is bonded to an aromatic nucleus. For this purpose, it is possible to use a heterogeneous catalyst containing ruthenium and optionally at least one metal of transition group I, VII or VIII. The support material used is, for example, aluminium oxide, silicon dioxide, titanium dioxide or zirconium dioxide, preferably aluminium dioxide or zirconium dioxide. Only a catalyst containing ruthenium on the support material aluminium oxide is given as an example, and not zirconium oxide.

EP 1337331 A1 discloses a process for catalytic hydrogenation of aromatic or heteroaromatic amines, wherein ruthenium acts as the active metal and the catalyst contains at least one additional metal of transition group I, VII, or VIII, and these are applied to a support material. Aromatic compounds used here include 4,4′-MDA and isomers thereof. EP 0111238 A1 also discloses a process for catalytic hydrogenation of 4,4′-MDA, characterized in that the hydrogenation is effected in the presence of supported ruthenium in the presence of nitrates and sulfates of the alkali metals and nitrates of the alkaline earth metals. A comparable process is disclosed in EP 1366812 A1, where support materials mentioned include aluminium oxide, silicon oxide, titanium oxide and zirconium oxide.

DE 101 19 135 A1 discloses a plant and a process for production of diaminodicyclohexylmethane (PACM) with a proportion of trans, trans-4,4′-diaminodicyclohexylmethane of 17% to 24%, where this is achieved by hydrogenation of diaminodiphenylmethane (MDA) in the presence of a pulverulent catalyst. The hydrogenation is conducted here in a continuously operated suspension reactor, in particular in a cascade of multiple series-connected suspension reactors, such that an MDA conversion of at least 95% is achieved, based on the amount of MDA used.

EP 1 519 912 B1 discloses a plant and a process for production of 4,4′-diaminodicyclohexylmethane (4,4′-HMDA), wherein by means of catalytic hydrogenation of a substance mixture comprising 4,4′-diaminodiphenylmethane (4,4′-MDA) as the main component and its mono-N-methyl derivative as a secondary component with elevated selectivity with regard to the hydrogenation of 4,4′-MDA in the presence of a heterogeneous hydrogenation catalyst at a temperature in the range from 50 to 220° C. and a hydrogen pressure in the range from 1 to 30 MPa is effected. For this purpose, EP 1 519 912 B1 suggests ending the hydrogenation prior to attainment of a conversion of 4,4′-MDA to 4,4′-HMDA of 99%, especially in the range from 90% to 98.9%.

Further processes for hydrogenation of organic compounds are disclosed by WO 2011/003899 A1 and WO 2009/090179 A1. What are disclosed here are processes for hydrogenation of aromatic amines with hydrogen in the presence of an Ru catalyst containing zirconium oxide support material inter alia.

Finally, EP 2 883 863 B1 (D1b) discloses a process and a plant for hydrogenation of 4,4′-methylenedianiline (MDA) and/or polymer MDA with hydrogen in the presence of a catalyst. A catalyst proposed here is ruthenium applied to a zirconium oxide support material. With regard to the reactor, EP 2 883 863 B1 refers to the reactor or the reactor concept of WO 2008/015135 A1 (D1a). Said WO 2008/015135 A1 discloses a continuous process and a plant for hydrogenation of diisononyl phthalate to diisononyl cyclohexane-1,2-dicarboxylate (DINCH), wherein DINP is hydrogenated as a mixture in an organic solvent with hydrogen at a pressure of up to 325 bar. What is proposed here is series connection of two fixed bed reactors each with an immobile fixed bed. In order to dissipate the heat of reaction from the two reactors, it is proposed that a substream of the stream of matter be recirculated and thereby cooled downstream of the second fixed bed reactor. A comparable reactor concept of series-connected fixed bed reactors for hydrogenation of is also known from EP 1 566 372 B1.

This concept is disadvantageous in that the circulation of a product substream can lead to increased formation of unwanted by-products and reduces plant performance, causing increased energy costs for the recycling and cooling of the recycling stream and making the operation of the plant and the process very energy-intensive overall.

As explained, the need for PACM, for example, with different proportions of the respective isomers is dependent on the intended use or the subsequent products. For example, PACM qualities with a low trans/trans content of 10% to 30% by weight are preferred in the field of amine and isocyanate crosslinkers, especially in the field of formulation of 2-component resins, and PACM qualities with a high trans/trans content exceeding 48% by weight are mainly used as regulator in polyamide compounds. The percentages by weight mentioned are based here on the PACM isomer mixture as such. The production of products with a low trans/trans content in particular is a process engineering challenge, since the thermodynamic equilibrium, as described in U.S. Pat. No. 3,636,108 A, is in the range of significantly higher trans/trans contents of up to 51.2% by weight. Furthermore, it is known from U.S. Pat. No. 2,606,925 A that the equilibrium of the PACM isomers is subsequently shifted in the direction of a higher proportion of trans/trans isomer by prolonged heat treatment.

### SUMMARY OF THE INVENTION

It is thus an object of the present invention to provide an improved plant and an improved process which is improved in terms of product conversion and energy efficiency and in particular enables the production of defined proportions of the respective isomers in the isomer mixture.

### BRIEF DESCRIPTION OF THE DRAWINGS

FIG. 1 shows a plant as a process flow diagram.

FIG. 2 shows a first embodiment of the plant with a first direct EC.

FIG. 3 shows a second embodiment of the plant with a further direct EC.

FIG. 4 shows a first embodiment of the reactor unit.

FIG. 5 shows a second embodiment of the reactor unit.

FIG. 6 shows a third embodiment of the plant with a further direct EC as integrated material-based EC.

FIG. 7 shows a fourth embodiment of the plant with a further direct EC as integrated material-based EC.

FIG. 8 shows a fifth embodiment of the plant with a further direct EC as integrated material-based EC.

FIG. 9 shows a sixth embodiment of the plant with a further direct EC as integrated material-based EC with series interconnection and circulation.

FIG. 10 shows a seventh embodiment of the plant with a circuit for onward conduction of media.

FIG. 11 shows an eighth embodiment of the plant with an alternative circuit for onward conduction of media to FIG. 10.

### DETAILED DESCRIPTION OF THE INVENTION

The object is achieved in accordance with the invention by a plant according to the features of embodiment 1 and a process according to the features of embodiment 16.

A plant is herein provided for continuous catalytic hydrogenation of methylenedianiline (MDA; reactant1) with a hydrogen donor (reactant2), especially a gaseous hydrogen donor, preferably hydrogen (H2), comprising a conditioning unit (104) for the reactants, a reactor unit (102) and a separation unit (106), wherein
the conditioning unit comprises (feed) conduits for reactants1, reactant2 and at least one solvent, at least one heat exchanger in at least one (feed) conduit, at least one mixer for mixing the reactants and/or at least one reactant with at least one solvent,
the reactor unit comprises at least one fixed bed reactor as main reactor with an immobile catalyst packing, where the at least one main reactor comprises a first flow pathway for the mixture of matter through the immobile catalyst packing, where the at least one main reactor is incorporated into a further flow pathway, and where the further flow pathway incorporates a heat exchanger for influencing the temperature level in the first flow pathway;
the separation unit comprises at least
a first separation stage comprising at least one apparatus for removing the solvent, where the at least one apparatus is connected downstream to at least one condenser via at least one (tops) conduit, and
a second separation stage comprising at least one apparatus for separation of at least one reactant and/or at least one by-product from the product, where the at least one apparatus is connected downstream of at least one condenser via at least one (tops) conduit, wherein
   the further flow pathway is a closed media circulation system for a heat transfer medium, at least a section of which runs outside the catalyst packing of the at least one main reactor for indirect heat transfer, where the media circulation system incorporates a heat exchanger.

The further flow path is especially connected to the other conduits of the plant and especially of the reactor unit such that no reactant or substance mixture can flow into it in regular operation.

By virtue of this further flow pathway for a closed media circulation system, advantageously incorporating a pump as delivery means, it is possible to achieve a significant increase in performance compared to WO 2008/015135 A1. Also surprisingly, a quasi-isothermal mode of operation of the main reactor can achieve a trans/trans ratio in the product of 22% by weight.

The plant is preferably used for continuous catalytic production of methylenebis(cyclohexylamine) as product, especially for production of 4,4′-diaminodicyclohexylmethane (PACM), of the formula (I)

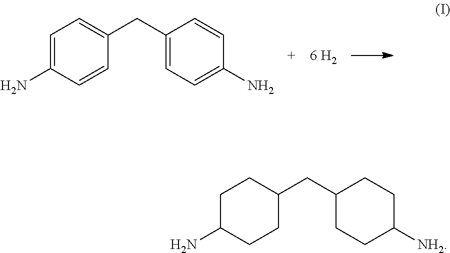 [SMILES: Nc1ccc(Cc2ccc(N)cc2)cc1 ; NC1CCC(CC2CCC(N)CC2)CC1]

In a further embodiment of the plant, it may be advantageous when the reactor unit comprises a first main reactor and at least one downstream series-connected further postreactor.

The postreactor is advantageously connected in series to the main reactor and has an immobile catalyst which may be physically identical or largely identical to that in the main reactor. The amount and spatial arrangement of the immobile catalyst, and also the flow pathway of the stream of matter within the postreactor, are selected such that a maximum of 20% of the molar conversion of MDA, advantageously a maximum of 15%, ideally a maximum of 10%, where the reaction is considered complete at 100% molar MDA conversion. The postreactor is advantageously operated as an adiabatic reactor, where, in a further embodiment of the plant, it may be advantageous when at least one heat exchanger is disposed in the conduit between the at least one main reactor and the postreactor.

As time advances, the catalyst activity decreases until replacement or regeneration is required. For this purpose, in parallel, the operating temperature is raised by controlling the cooling circuit, i.e. in particular by cooling to a lesser degree via the heat exchanger incorporated in the cooling circuit in order to keep conversion and selectivity at a largely constant level overall. As a result, the isomer ratio is shifted, toward higher trans/trans contents in the product. It has been found here to be very advantageous that the temperature in the feed of the postreactor can be controlled autonomously by means of the upstream heat exchanger, in particular in the same sense. Thus, as the operating temperature of the main reactor rises over the service life of the catalyst, the feed temperature in the stream of matter of the postreactor is lowered.

With the separate postreactor, especially the adiabatic postreactor, an optimized control of the process and the plant has been enabled, whereby selectivity becomes possible because of the variance of the inlet temperature of the postreactor from the main reactor (outlet). Typically, the inlet temperature of the postreactor may be provided at the same or a slightly lower temperature, which may be up to 30° C. below the outlet temperature of the main reactor. Thus, at least temporarily, an increase in temperature in the (adiabatic) postreactor of 20° C. to 40° C., typically of 20° C. to 30° C., may be allowed, and hence the desired product quality (isomer ratio) can be specifically adjusted. In this way, a very low and a very exact proportion of trans/trans isomers in the isomer mixture can be established. The main reactor can be operated at a very low temperature level such that the trans/trans content of the PACM in the stream of matter (outlet of the main reactor) is about 13% to 20% by weight, with about 13% being achievable in the case of a new or regenerated catalyst and 20% by weight in the case of a catalyst after prolonged use (shortly before replacement/regeneration). Depending on the main reactor phase, it may be useful at least temporarily if the inlet temperature of the postreactor is 5 to 20° C. higher than that of the main reactor.

Even though the main reactor is referred to in the present context as being “isothermal”, this ideal state is only achieved to a limited degree in industrial use, and so a temperature gradient of about 5 to 10° C. in radial direction and also in flow direction develops within the main reactor owing to incomplete heat dissipation.

The postreactor is charged via the upstream heat exchanger with a somewhat higher temperature so as to result in the desired residual reaction and isomerization to give the final desired trans/trans ratio of 17% to 23% by weight, for example. The postreactor with the weakly exothermic residual reaction therein is operated in a largely adiabatic manner, although this should not be understood in the ideal sense.

From time t₀, the start of the process after renewal or regeneration of the catalyst, to time t₄, the end of the process determined by renewal or regeneration of the catalyst, the operating temperature of the main reactor can be increased and the temperature in the stream of matter in the inlet (feed) to the postreactor can be maintained or lowered. Advantageously, the plant is designed such that the temperature can be increased or lowered continuously and/or stepwise.

The main reactor is operated here isothermally or largely isothermally, and the postreactor adiabatically or largely adiabatically. At time t₀, the start of process, the catalyst in the postreactor may also have been renewed or regenerated as well as the main reactor. Advantageously, the cycle for regeneration of the postreactor is determined autonomously and independently of the main reactor, especially when the abovementioned interaction of the two reactors no longer enables the production of the desired low trans/trans content in the PACM.

The liquid stream of matter coming from a flash separation tank, referred to hereinafter as separation tank (also called flash vessel in some cases), is directed via a conduit to the first separation column within the first separation stage of the separation unit. It is a feature of the separation tank that the incoming stream of matter is separated into a vapour phase (solvent, solvent-rich) and a liquid phase (solvent-depleted) by expansion (lowering the pressure) and that both phases are present in the separation tank in regular operation. The separation tank may additionally have a bottoms circulation system with integrated heat exchanger or be connected thereto in order to increase the removable vapour content beyond the pressure-related fraction by heating the liquid phase. Furthermore, a separation tank may comprise internals or random packings, in order in particular to prevent entrainment of droplets that have only been incompletely depleted of solvent, if at all. Advantageously, in one embodiment of the plant, it may be the case that a pressure control unit is provided in the conduit from the separation tank to the first column. This allows the reactor unit to be operated at a first, high pressure level, and the first separation stage of the separation unit at a second, lower pressure level. What is thus meant in the present context by separation tank (flash vessel) is a device whereby a phase separation is caused essentially by expansion. What is meant here by separation column, by contrast, is an apparatus in which a separation into a vapour phase and liquid phase takes place essentially by supply of energy, in particular by incorporating a tops circulation system in which at least a portion of the liquid condensed out is directed back into the column at the top.

Advantageously, the mixing unit is formed in two parts and comprises, for example, a mixing apparatus and a gas saturator. The mixing apparatus may especially be a dynamic or static mixer suitable for intimate association of MDA (reactant1) with the solvent. The gas saturator may especially be a small column or a tank in which there are suitable internals for intensive dissolution of the H2 gas supplied under a high pressure in the MDA/solvent stream of matter and/or for homogeneous distribution thereof prior to entry into the main reactor. The H2 gas pressure is advantageously 70 bar to 100 bar.

The term “immobile catalyst packing” or “immobile catalyst” refers to any form of a local catalyst that does not flow or move with the stream of matter, such as, in particular, catalyst beds (pellets or coated carrier bodies) or fixed installations coated with catalyst material. Advantageous internals having a catalyst coating may be, for example, grids, plates or other bodies arranged in the main reactor.

In the present context, an “XY unit” and/or “XY stage” always also means at least one corresponding apparatus, device or the like which is included in the respective unit or stage. This means, for example, a mixing unit/stage comprising this at least one mixer/mixing device.

What is meant in the present context by “heat exchange” or “heat exchanger” is always indirect heat exchange and corresponding designs with closed material and media conduits, in the absence of any explicit description to the contrary.

The “condensation unit” of the first separation stage refers to a single heat exchanger or a group of heat exchangers which are used for at least partial condensation and/or cooling of the fractions of light boilers derived via the top conduit(s). A “condensation unit” referred to as such need not be a (closed) structural unit. Thus, to some degree, the term “condensation unit” and a single “heat exchanger” are also used synonymously. A “condenser” means a heat exchanger that has a cooling effect on a (vapour) conduit and is intended to cause at least partial condensation of the vapour in the corresponding conduit. However, such a name should not be understood in a limiting manner, because all media streams, reactant streams and streams of matter are considered in the present context of the EC, such that, for example, a condenser can serve as a heat source for a coupled downstream heat exchanger. The final understanding of what is meant must therefore always be taken from the respective textual context.

The stage referred to as “first separation stage” is determined in particular, and has corresponding apparatus and conduits, in order to (specifically) separate the solvent from the product-rich stream of matter and advantageously also to return it to use in the reactor unit and/or the conditioning unit. The first separation stage is determined in that at least 80%, ideally at least 90%, of the solvent is separated off. In an analogous manner, the stage of the separation unit referred to as the “second separation stage” means that it is thus determined, and has corresponding apparatuses and conduits, in order to separate the product from by-products and reactants, in particular MDA, and purify the product. In this case, the first and second separation stages may not be entirely strictly separated, and there may be an overlap region or an apparatus in the transition region in which both solvent and at least one by-product or at least one reactant is separated from the product. In the present context, what is meant by the “removal of solvents” in the first separation stage and “separation of at least one reactant and/or at least one by-product from the product”, where the product may especially be PACM, is that the removal/separation does not mean an absolute delimitation of the separation stages, but affects “essentially” only the substance(s) mentioned in each case.

What is meant in the present context by the term “reactant mixture” is the mixture of matter present on entry into the (first) main reactor, i.e. the mixture of all reactants, solvents, auxiliaries etc. in and downstream of the at least one reactor, the flowing mixture of matter in any degree of reaction or subsequent purification is referred to as “stream of matter” or “mixture of matter”, in some cases with addition of adjective descriptions such as “product-rich” or “solvent-rich”. However, the physical composition of the stream of matter at each site in the plant is also obvious to the skilled person by virtue of that site in the plant and upstream plant components, and in particular the process engineering apparatuses of the plants. The pure substances, for example the PACM product, and the LB and HB by-products, are named and identified separately. “LB” here stands for “low boilers”, a valuable mixture of matter that is separated separately from the stream of matter and the product and has a low boiling point of about 240° C. to 290° C. Analogously, “HB” stands for “high boilers”, a valuable mixture of matter that is separated separately from the stream of matter and from the product and has a high boiling point of >350° C.

What are described in particular in the present context are a plant and a process for production of methylenebis(cyclohexylamine) as product, especially for production of 4,4′-diaminodicyclohexylmethane (PACM), by catalytic hydrogenation of methylenedianiline. The primary product here is PACM, which is hydrogenated from 4,4′-diaminodiphenylmethane (4,4′-MDA; primary fraction of reactant1), especially with the low trans/trans isomer ratio mentioned, and so the plant and the process serve, and are suitable, in particular for production of 4,4′-diaminodicyclohexylmethane (PACM) by continuous catalytic hydrogenation of 4,4′-diaminodiphenylmethane (4,4′-MDA). The additional fractions of 2,4′-MDA and 2,2′-MDA of the MDA are converted at least in small proportions in parallel to reaction products, where these generally remain in the product mixture. In addition, other by-products can advantageously likewise be purified and separated off by this process or this plant in the second separation stage of the separation unit, especially in at least one separation column. These are secondary (valuable) products, herein described as and meaning essentially high boilers (HB) and low boilers (LB).

The solvent is advantageously from the following group of substances: cyclohexane, dioxane, tetrahydrofuran (THF), cyclohexylamine, dicyclohexylamine, methanol, ethanol, isopropanol, n-butanol, 2-butanol, 2-methoxy-2-methylpropane (MTBE) or methylcyclohexane or a mixture thereof. Advantageously, the solvent, especially THF, is fed in excess into the MDA, such that the ratio of the mass flow rates of solvent, especially THF, to MDA at the inlet of the main reactor is advantageously in the range from 1.0 to 8.0, especially in the range from 2.0 to 7.5.

In the present context, different forms of energy coupling and heat transfer are described, which are defined as follows:

Overall, it may be advantageous when the first separation stage of the separation unit comprises at least one separation tank connected via a conduit to the main reactor, the postreactor or the last main reactor in flow direction, where the separation tank comprises a tops outlet, a base/bottoms outlet and a heatable bottoms circulation system having at least one heat exchanger, in which the (tops) conduit connected to the tops outlet incorporates the at least one condensation unit. In addition, a collection vessel and/or a connecting unit/node with/into the (return) conduit for the solvent may be disposed downstream of the condensation unit.

The stream of matter coming from the separation tank is directed via a (feed) conduit to the first separation column in the second separation stage of the separation unit. Advantageously, in one embodiment of the plant, it may be the case that a pressure control unit is provided in the (feed) conduit from the separation tank to the first column. This allows the reactor unit to the operated at a first, high pressure level, and the first separation stage of the separation unit at a second, lower pressure level.

It may advantageously be the case that at least one EC is provided, incorporating at least one of the following heat exchangers:
the at least one condenser in the first separation stage, especially the condenser in the (tops) conduit of the one separation tank,
the at least one condenser in the second separation stage, especially a condenser in a separation column in the second separation stage,
the at least one heat exchanger of the media circulation system of the at least one main reactor.

What is meant here by energy coupling (EC) is integrated energy coupling or a direct energy coupling, where
i) integrated EC means integrated energy coupling, subdivided into
   a. integrated material-based energy coupling (integrated material-based EC) of at least two streams of matter in a heat exchanger in indirect heat exchange, i.e. structural integration in a single heat exchanger (apparatus), meaning that, where two heat exchangers were formerly provided, both heat transfer functions of a structural unit (heat exchanger) are integrated,
   b. integrated media-based energy coupling (integrated media-based EC) of at least two heat exchange media in a heat exchanger in indirect heat exchange, meaning structural integration in a single heat exchanger (apparatus).
ii) direct EC (direct energy coupling), also called series EC, means interconnection of at least two structurally separate heat exchangers, subdivided into:
   a. series integrated EC, where a stream of matter (integrated material-based EC) or media stream (integrated media-based EC) from a first heat exchanger is passed on to a downstream second heat exchanger for the next integrated EC, in order each to act on a (different) stream of matter or (different) media stream,
   b. series material-based EC, wherein a stream of matter from a first heat exchanger is passed to a downstream second heat exchanger in order each to act on a (different) media stream and/or
   c. series media-based EC, where a media stream from a first heat exchanger is passed on to a second downstream heat exchanger in order each to act on a (different) stream of matter.

In the present context, however, EC does not mean a (standard) solitary heat exchanger without further (thermal) interconnection in which there is indeed (only) one reactant stream or stream of matter that is heated or cooled by (only) one heat exchange medium. Furthermore, in the present context, “further stream of matter” means a stream of matter at another plant site, in a different physical composition and/or at a different temperature level, and “a further media stream” should be understood analogously.

In an advantageous variant of this embodiment of the plant, it may be the case that the plant (100) comprises at least one series interconnection of two heat exchangers to the integrated material-based EC, wherein a stream of matter from the first heat exchanger in integrated material-based EC is passed onward in series interconnection to the second downstream heat exchanger for further integrated material-based EC.

In a further advantageous embodiment of the plant, it may be the case that at least one heat exchange circuit takes the form of a direct EC (series EC) incorporating at least two heat exchangers for series matter-based EC or media-based EC. The heat exchange circuit is advantageously a closed circuit. Overall, it may be advantageous in the case of EC and circuits as EC, as required, to provide heat exchangers as on-demand or control heat exchangers with independent coolants or heat sources in particular, in order to be able to ensure thermodynamic energy open-loop and closed-loop control of the system as a whole and to achieve an additional degree of control freedom. These on-demand or control heat exchangers are generally distinctly smaller in terms of construction, especially in the case of integrated material-based EC, than the former integrated heat exchanger that has been omitted according to integrated material-based EC.

In a further advantageous embodiment of the plant, it may be the case that at least one of the following conduits in the condenser is incorporated in the (tops) conduit of the separation tank as heat source for integrated material-based EC as follows:
i) the (feed) conduit to the main reactor,
ii) the (feed) conduit (116) to the postreactor,
iii) the (feed) conduit to the first separation column, where a pressure control unit is disposed in the (feed) conduit upstream of the condenser,
iv) at least one further heat exchanger for integrated material-based EC is incorporated downstream of an incorporation of a conduit according to i), ii) or iii) in the condenser and upstream of a collection vessel.

In a further advantageous embodiment of the plant, it may be the case that the plant comprises a serial media-based EC as circuit with a plurality of conduit sections, incorporating at least the following apparatuses and/or conduits: the collection vessel downstream of the separation tank, the (tops) conduit as a conduit section, in particular a first conduit section, the (feed) conduit to the separation tank as a further conduit section, in particular a last conduit section. In this embodiment, the solvent as heat exchange fluid passes through the (heat exchange) circuit, and the first flow pathway of the main reactor is a conduit section of the circuit. The naming of conduit sections as first or last conduit section indicates a flow direction in the circuit, but is not intended otherwise to constitute any restriction.

In one variant of this embodiment of the plant, it may be the case that the (feed) conduit to the first separation column of the first separation stage in the condenser is incorporated into the (heat exchanger) circuit in the (tops) conduit of the separation tank for integrated material-based EC, where the (tops) conduit serves as heat source for the (feed) conduit. It is particularly advantageous here when a pressure control unit is disposed upstream of the condenser in the (feed) conduit.

In a further advantageous embodiment of the plant, it may be the case that the media circulation system of the main reactor is incorporated in the heat exchanger as heat source by the (feed) conduit to the first separation column for EC. A pressure control unit is disposed here in the (bottoms) conduit upstream of the heat exchanger of the media circulation system. The temperature is thus at a very advantageous level in the (feed) conduit to the first separation column, such that a heat exchanger incorporated downstream can absorb correspondingly large amounts of heat at an advantageously low temperature level.

In the present context, a multipart noun between parentheses is frequently used, for example (feed) conduit, (bottoms) conduit, (tops) conduit etc. The nouns between parentheses serve for the illustration and easier linguistic comprehension of what is meant, but are not supposed to constitute a limitation, since, for example, a (tops) conduit of a vessel, in the downstream direction, constitutes a (feed) conduit connected, for example, to a corresponding vessel. Furthermore, “flow pathway”, “stream of matter”, “media stream”, “reactant stream” are used partly as a synonym for the associated conduit, which is done in particular for the descriptions of the plants, the associated apparatuses, components and elements and the mutual arrangement thereof. Similarly, an EC always also means a corresponding apparatus, such as a heat exchanger, condenser, etc.

In a further advantageous embodiment of the plant, it may be envisaged that at least one of the following conduits is incorporated in a condenser of a separation column of the second separation stage in the respective (tops) conduit as heat source for integrated material-based EC as follows:
i) the (feed) conduit to the main reactor,
ii) the (feed) conduit to the postreactor,
iii) the (feed) conduit to the first separation column of the first separation stage, where a pressure control unit is disposed in the (feed) conduit upstream of the condenser of the respective separation column.

In an advantageous embodiment of the plant, it may be the case that the media conduit:
i) leads from the condenser of the separation tank of the separation unit as heat source to at least one of the following heat exchangers of the reactor unit or the conditioning unit:
   heat exchanger in a (feed) conduit to the main reactor and/or an apparatus disposed upstream of the main reactor, especially mixer or saturator,
   heat exchanger in the (feed) conduit to the postreactor and/or
   heat exchanger in the (feed) conduit to the first separation column of the separation unit, and where at least two of the heat exchangers operating as direct EC may be connected in parallel to one another;
ii) leads from the (cooling) heat exchanger of the reactor as heat source to at least one of the following heat exchangers:
   heat exchanger in a (feed) conduit to the main reactor and/or an apparatus disposed upstream of the main reactor, especially mixer or saturator,
   heat exchanger in the (feed) conduit to the first separation column of the separation unit, and where at least two of the heat exchangers working as direct EC may be connected in parallel to one another; and/or
iii) leads from one of the (cooling) heat exchangers in the tops circulation system of an apparatus of the separation unit to at least one of the downstream (heating) heat exchangers in the feed or bottoms circulation system of an apparatus of the separation unit, especially
   from the heat exchanger of the separation columns of the second separation stage to the heat exchanger in the (feed) conduit to the first separation column,
   from the condenser in the tops circuit of the separation column that separates off first product, especially PACM product, as heat source to the heat exchanger in the (feed) conduit to the first separation column,
   from the condenser in the tops circuit of the separation column as heat source to the heat exchanger in the bottoms circulation system of the separation tank.

In the present context, “media conduit” means a conduit in which a heat exchange fluid flows, which is neither a reactant mixture nor a mixture of matter. Typically, the heat exchange fluid is water, water vapour, a brine or an oil. “Media circuit” should also be understood analogously in the present context.

In a further embodiment of the plant, it may be advantageous when the separation unit in the first separation stage comprises at least one pressure control unit and a separation tank with a condensation unit for the solvent in the (feed) conduit to the separation tank. It is advantageously possible here for a (return) conduit for the solvent to lead from the first separation stage, in particular a collection vessel, to the conditioning unit.

The liquid stream of matter coming from the separation tank is directed via a (feed) conduit to the first separation column in the first separation stage of the separation unit. Advantageously, in one embodiment of the plant, it may be the case that a pressure control unit is provided in the (feed) conduit from the separation tank to the first column. In this way, the reactor unit may be operated at a first, high pressure level, and the first separation column of the first separation stage at a second, lower pressure level, where the separation tank may be operated at an intermediate level.

Furthermore, it may be advantageous when a heat exchanger is provided upstream of the first separation column, especially also downstream of the pressure control unit or between the pressure control unit and the first separation column. In an advantageous embodiment, media conduits are provided in order to interconnect, in a series media-based EC, the (forward) heat exchanger of the postreactor or the condenser in the tops circuit of the separation tank with the (forward) heat exchanger of the first separation column.

By direct energy input, for example by means of superheated steam, in a first step, the stream of matter coming from the main reactor (feed to the first separation column) can be heated by about 5 to 20° C. in a (forward) heat exchanger which has an inlet-side temperature level of about 85 to 95° C. downstream of the upstream pressure control unit. The outgoing heating medium, a vapour-condensate mixture, can be directed downstream of a series-coupled (forward) heat exchanger of the postreactor. The advantage of such a solution is that the first separation column still has a bottoms circulation system and a (bottoms) heat exchanger incorporated therein, which, if required, can cover the complete energy requirement of the first separation column. Thus, the coupling can result in optimal energy exchange with the downstream (forward) heat exchanger (series media-based EC) of the postreactor on the media side, while the remaining amount of energy can flow to the first separation column as an energy saving.

In a particularly advantageous embodiment, an energy coupling is provided in order to operate the condenser downstream of the separation tank, a condenser of a separation column of the second separation stage or the (circulating) heat exchanger in the media circuit of the at least one main reactor interconnected in heat exchange with the (forward) heat exchanger of the first separation column. The energy coupling can be effected by conduction of media and a series interconnection of the respective heat exchangers or by integrated energy coupling in a (structurally) single heat exchanger. Integrated energy coupling has the advantage, if spatially implementable within the plant, that only a temperature gradient for heat transfer has to be overcome. It is thus possible by energy transfer to achieve a parallel rise in the (feed) stream of matter upstream of the first separation column of the first separation stage, which is at a temperature level of about 85 to 95° C. downstream of the upstream pressure control unit in the (feed) conduit.

Since the main reactor, in the course of operation, is operated at a constantly rising temperature in order to compensate for the falling catalyst activity, a constantly rising amount of energy can be released to the (feed) stream of matter upstream of the first separation column in parallel. As a result, there is likewise a steady drop in the energy demand in the bottoms circuit, or the heat exchanger of the first separation column incorporated therein.

In one embodiment of the plant, it may be advantageous that the reactor unit comprises a first main reactor and at least one downstream series-connected postreactor. The particular advantage of the postreactor and its inlet-side temperature control of the stream of matter is that optimized control of the selectivity of the proportions of isomers is enabled in this way, because of the preferably higher inlet temperature that differs from the main reactor.

Advantageously, the postreactor is also a fixed bed reactor, or reactor with an immobile catalyst, for example a catalyst bed or catalyst-coated internals.

Advantageously, the immobile catalyst comprises ruthenium, has been doped with ruthenium or is formed therefrom. In an advantageous process variant, especially for achieving a low proportion of trans/trans isomers in the isomer mixture, the main reactor is at a temperature of 90 to 140° C., ideally 95 to 135° C.

It has been found to be particularly advantageous when the ratio of the catalyst masses of the main reactor to the postreactor is in the range from 1.2 to 2, preferably 1.3 to 1.4 and ideally 1.35. It has been found that, surprisingly, it is sufficient to regulate the highly exothermic reaction at the start of the reaction in the main reactor by means of an intensive cooling circuit, and only to adjust the feed temperature in the inlet to the postreactor such that the moderate temperature rise of about 30 to 35° C. in the postreactor from the inlet to the outlet has only a limited and readily reproducible influence on the trans/trans isomer content in the stream of matter or in the product, as already set out.

It may be especially advantageous when the main reactor is a fixed bed reactor comprising
a first flow pathway for the reactant mixture or mixture of matter and
a further (closed) flow pathway, i.e. a media circuit for a heat exchange medium, where the second flow pathway incorporates two heat exchangers for indirect heat exchange:
a (main) heat exchanger operating as a cooler and
a (secondary) heat exchanger operating as a heater.

It has been found to be very effective and advantageous to operate the same further flow pathway, or media circulation, for preparation and startup of the main reactor by means of the (secondary) heat exchanger and to implement the cooling of the main reaction in producing operation by the main reactor by means of the (main) heat exchanger.

In a further embodiment of the plant, it may be advantageous that
the reactor unit comprises a further main reactor in the form of a fixed bed reactor comprising
   a first flow pathway for the mixture of matter and
   a further (closed) flow pathway, i.e. a (cooling) media circulation system for a heat exchange medium, and where a valve unit is provided upstream of the two main reactors in the (feed) conduit, by means of which the volume flow rate of the reactant mixture is divisible, conductable and/or completely switchable between the first main reactor and the further main reactor, and wherein the two main reactors are connected (in a thermally conductive manner)
   each to one heat exchanger or
   collectively to one heat exchanger
for the further (closed) flow pathway.

In this case, the two main reactors connected in series are identical or essentially identical in design. In particular, the two main reactors have such dimensions and/or corresponding internals such that the same or substantially the same mass and/or volume of catalyst is present or accommodatable.

The advantage of these two main reactors is that further thermal decoupling of the first reaction phase with very strong exothermicity, the second reaction phase with medium exothermicity and the postreaction with very low exothermicity is possible. Further advantages are that, in the case of the same plant performance, the individual main reactor has smaller dimensions and can therefore be thermally controlled more easily and more homogeneously. At the same time, plant performance is increased because, in the case of maintenance, for example a catalyst changeover, the plant does not have to be shut down completely. For this purpose, the two main reactors are interconnected in such a way that the mixture of matter can also flow through each alone, bypassing the respectively other main reactor (bypass 1).

The hydrogenation in the (isothermal) main reactor with significant cooling allows significant limitation of temperature-induced isomerization. In the (adiabatic) postreactor, a sufficient temperature level is then established in a controlled manner in the incoming stream of matter, especially via heat exchange, and hence isomerization is permitted to specifically afford an on-spec trans/trans content in the product. In a purely isothermal mode of operation of a single main reactor without a postreactor, what would at first be obtained would be excessively low trans/trans contents and a high proportion of unconverted MDA. By virtue of the option of allowing controlled adiabatic hydrogenation of the stream of matter, it is possible to advantageously control the evolution of temperature and hence isomerization in the postreactor.

In a further-improved variant, the interconnection is such that the postreactor can be bypassed in the case of operation of at least one main reactor (bypass 2), but can especially be bypassed in the case of operation of the two main reactors connected in series. In the bypass 2 interconnection variant, the main reactor through which the flow passes second in flow direction at least temporarily assumes the function of the postreactor, such that the plant can be operated without or largely without a drop in performance and/or changes in product quality, especially in the respective proportion of isomers in the isomer mixture.

In a further embodiment of the plant, it may be advantageous that at least one common heat exchanger is disposed in the conduit between the at least one main reactor and the second main reactor. This common heat exchanger is disposed in a central branch of both coolant circuits. The guiding and interconnection of the conduits here is such that, downstream of the common heat exchanger, the cooling medium is first introduced into the first of the two main reactors in which the more strongly exothermic reaction proceeds. The already heated cooling medium is then fed via a conduit into the downstream second main reactor, such that the latter is charged with a feed temperature different from the first main reactor.

The advantage is that it was observed that, surprisingly, in particular, very exact control of the first, highly exothermic reaction phase is crucial for product quality, and so it is possible to dispense with the construction work and control complexity involved in a further, completely independent second cooling circuit. Another reason for this is in particular because complete reaction can be ensured and controlled via control of the feed temperature of the postreactor connected in series downstream of the two main reactors; in particular, the desired low trans/trans isomer content can be established.

In a plant variant with improved controllability, it may be the case that a heat exchanger (post cooler) which is switchable and controllable as required is disposed in the respective (cross-) conduit of the cooling circuits by which the coolant outlet of the first main reactor is connected to the coolant inlet of the second main reactor.

The bottoms circulation system of the separation tank is operated at a temperature of 130° C. to 150° C., ideally 135 to 145° C. The great advantage of the separation tank, which is very simple in terms of construction and control, is that the boiling temperature of the mixture is likewise lowered by the lowering of pressure, such that heating in the separation tank is only necessary up to that lowered boiling temperature. Furthermore, by virtue of this measure, about 90% of the solvent present in the reactant mixture, in particular THF, is already removable. There is no need for this purpose for a tops circuit or return stream, as in a column. The stream of matter passed onward to the first separation column thus advantageously has only a remaining solvent concentration of about 20% to 40% by weight, ideally 25% to 35% by weight. It is thus possible for the first separation column to be of smaller design, and operable in a more energy-saving manner owing to the lower mass of the stream of matter.

In this way, the temperature of the stream of matter is additionally lowerable more quickly, and so any rise in the proportion of trans/trans isomers of the product, especially the PACM product, in the stream of matter is prevented or reduced.

The solvent-conducting (return) conduit is connected to the conditioning unit and/or to at least one suitable collection vessel.

Finally, in an advantageous embodiment of the plant, at least one heat exchange circuit may be included in the form of a series integrated material-based EC having a plurality of conduit sections incorporating at least the following apparatuses and heat exchangers and connected via at least one of the conduit sections,
i) as heat exchanger and as heat source for the heat exchange circuit
   the heat exchanger in the media circulation system of the main reactor or
   the (tops) heat exchanger in the (tops) conduit of the separation tank, each in integrated material-based EC with at least one of the following conduits with the reactant or stream of matter conducted therein as heat sink for the heat exchange circuit:
a) the (feed) conduit to the first separation column downstream of the pressure control unit,
b) the (feed) conduit to the postreactor, especially on the suction side of a pump incorporated therein,
c) the (feed) conduit upstream of the reactor, especially a mixing vessel upstream thereof, or the (return) conduit to the conditioning unit upstream of the mixer;
ii) as apparatus, at least the main reactor, especially the main reactor and the postreactor, at least one separation tank, at least one collection vessel of the first separation stage, and where
iii) the following conduits are integrated as conduit sections of the circuit:
a) a (the first) conduit section corresponding to the (tops) conduit of the at least one separation tank and/or
b) a (last) conduit section corresponding to the (feed) conduit to the at least one collection vessel in the second separation stage.

The invention further encompasses a process for continuous catalytic hydrogenation of methylenedianiline (MDA; reactant1) with a hydrogen donor (reactant2), especially a gaseous hydrogen donor, preferably hydrogen (H2), wherein production is effected by means of an industrial plant, wherein the plant is designed according to at least one of the working examples and variants described herein, and wherein the main reactor is operated at a temperature in the range of 80° C. to 150° C.

In an advantageous embodiment, by means of energy coupling (EC):
i) energy is transferred from the condenser of the separation unit, here the condenser in the tops circuit of the separation tank, as heat source for at least one heat exchanger of the reactor unit, the conditioning unit and/or the separation unit by means of integrated EC or direct EC in the range of the available amount of heat, especially in the range of 20% to 40%;
ii) heat exchanger of the (cooling) media circulation system for the main reactor of the reactor unit as heat source for at least one heat exchanger of the reactor unit, the conditioning unit and/or the separation unit transfers energy by integrated EC or direct EC in the range of 5% to 30% of the available amount of heat; especially in the range of 10% to 20%, and/or
iii) heat exchangers of the separation unit exchange energy by means of integrated EC or direct EC in the range of 5% to 100% of the available amount of heat, especially in the range of 30% to 90%.

In a further embodiment of the process, it may be an advantage that, in addition to the at least one main reactor, at least one postreactor comprising an immobile catalyst is also provided, where the at least one main reactor and the at least one postreactor are operated at the same or essentially at the same pressure.

In a further embodiment of the process, it may be an advantage that the temperature of the reactant stream at the inlet of the main reactor is 90 to 140° C., ideally 100 to 135° C.

In a preferred embodiment of the process, it may be the case that continuous catalytic production of methylenebis(cyclohexylamine) is effected, especially production of 4,4′-diaminodicyclohexylmethane (PACM), preferably 4,4′-diaminodicyclohexylmethane (PACM) with low proportions of trans/trans isomers. In a preferred process variant, continuous catalytic production of methylenebis(cyclohexylamine) is effected, especially production of PACM, of the formula (I)

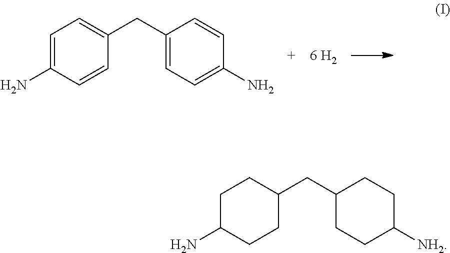 [SMILES: Nc1ccc(Cc2ccc(N)cc2)cc1 ; NC1CCC(CC2CCC(N)CC2)CC1]

In a further embodiment of the process, a further advantage may be that the at least one main reactor is operated at a pressure in the range from 60 bar to 120 bar, ideally in the range from 70 to 100 bar.

In a further advantageous process regime, it may be the case that the pressure in the main reactor is 80 to 90 bar. Particular preference is given to a pressure of about 85 to 95 bar.

In a further embodiment of the process, a further advantage may be that, from time t₀, the start of the process after renewal or regeneration of the catalyst, to time t₄, the end of the process determined by renewal or regeneration of the catalyst, the operating temperature of the main reactor is increased and the temperature in the stream of matter in the inlet (feed) to the postreactor is maintained or lowered, where the increasing or lowering of the temperature is linear and/or stepwise.

The plant variant mentioned thus enables a process (sequence) until attainment of a low trans/trans content in the PACM of 17% to 25% by weight over the time.

In a further embodiment of the process, a further advantage may be that the MDA (reactant1) comprises a mixture of the at least two isomers: 4,4′ MDA, 2,4′ MDA and 2,2′ MDA. The MDA (reactant1) is advantageously a mixture, where the mixture comprises the following monomers: 4,4′ MDA, 2,4′ MDA and 2,2′ MDA, where the proportion of 4,4′ MDA is advantageously in the range from 75 to 98 mol %, preferably 85 to 95 mol %, ideally about 90 mol %. The proportion of 2,4′ MDA in the reactant mixture is advantageously 7 to 15 mol %, preferably 8 to 12 mol %, ideally 9 to 10 mol %.

Ideally, the proportion of 4,4′ PACM (trans/trans PACM) is in the range from 15% to 30% by weight, ideally 16% to 25% by weight.

Overall, it is advantageous when the reaction step in the main reactor is implemented isothermally or largely isothermally and the reaction step in the postreactor is implemented adiabatically or largely adiabatically. At time t₀, the start of process, the catalyst in the postreactor may also be in renewed or regenerated form as well as the main reactor. Advantageously, the cycle for regeneration of the postreactor is determined autonomously and independently of the main reactor, especially when the abovementioned interaction of the reaction components in the two reactors no longer enables the production of the desired low trans/trans content in the PACM.

In an advantageous embodiment of the process, it may be the case that the temperature of the reactant stream at the inlet of the main reactor is 90 to 140° C., ideally 100 to 135° C., preferably 105 to 115° C.

In a further advantageous embodiment of the process, it may be the case that the pressure in the main reactor is 70 to 100 bar, ideally 80 to 90 bar.

In a further advantageous embodiment of the method, it may be the case that the temperature at the main reactor inlet corresponds essentially to the temperature at the postreactor inlet, where essentially means a range or difference of +/−10° C. and/or the pressure at the main reactor inlet corresponds essentially to the pressure at the postreactor inlet, where essentially means a range or difference of +/−5 bar.

Overall, all aspects, advantages and executions relating to plants or mentioned in association with the description thereof are also intended to be applicable identically or analogously to the process, and vice versa, unless stated otherwise and/or there is a technical impossibility associated with analogous application.

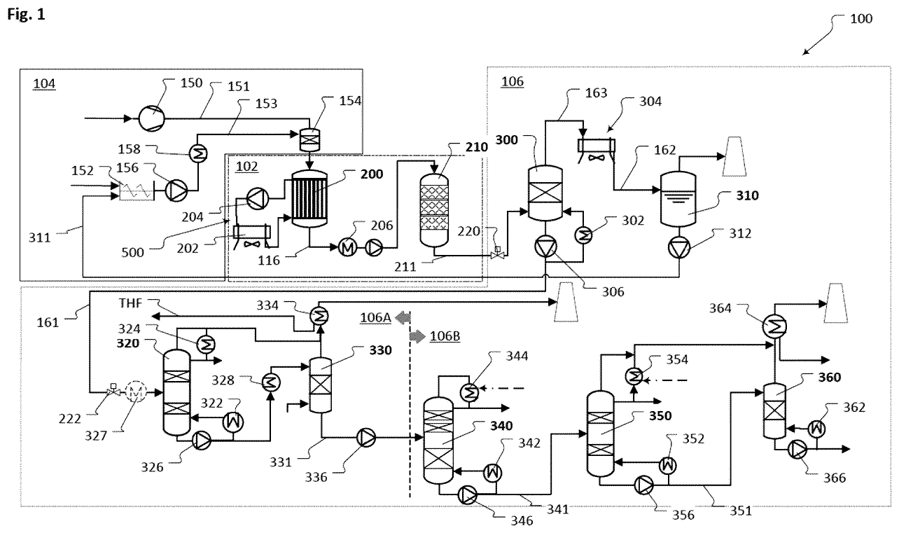

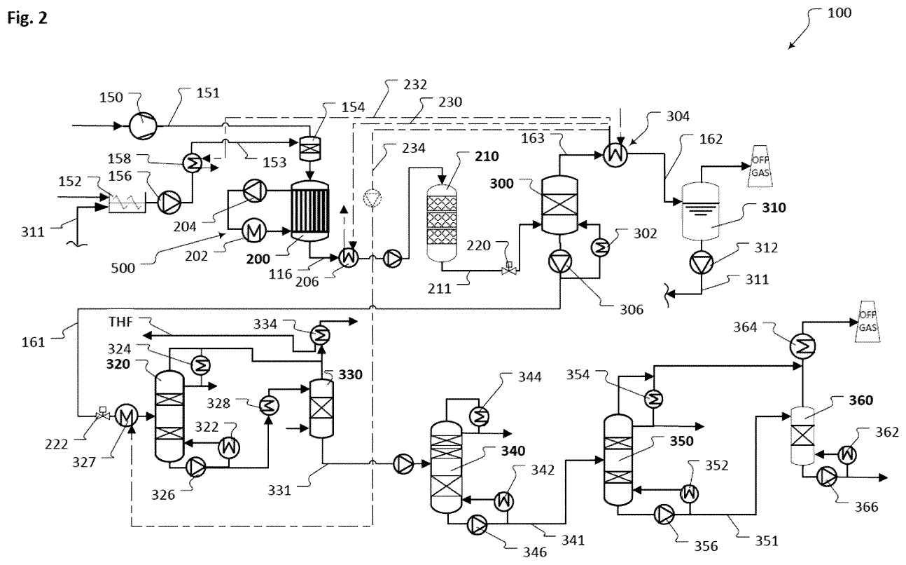

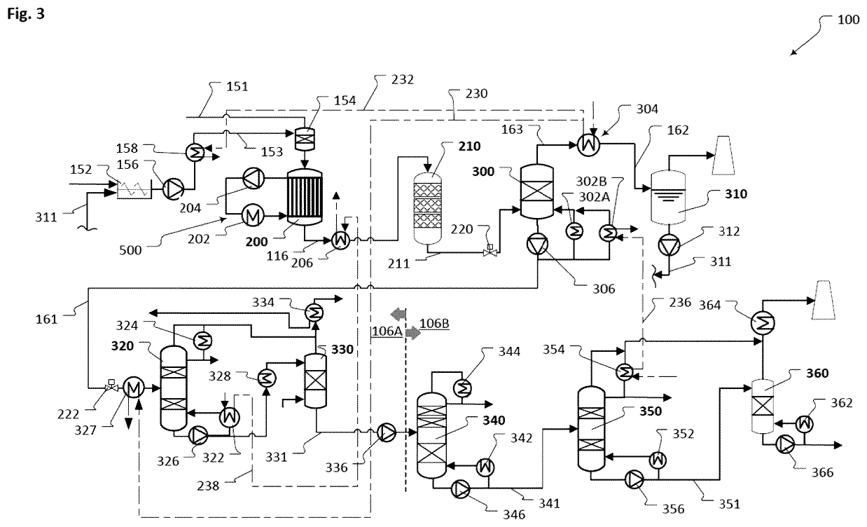

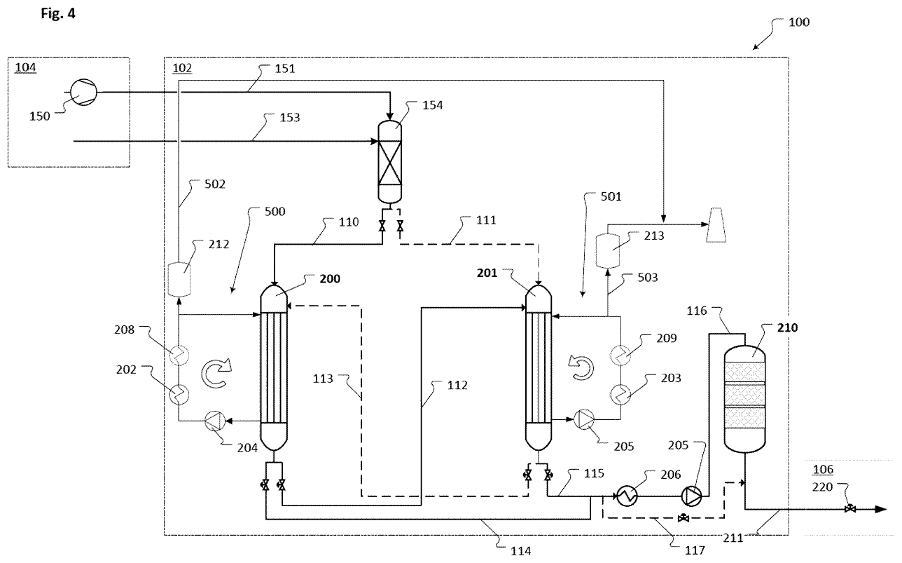

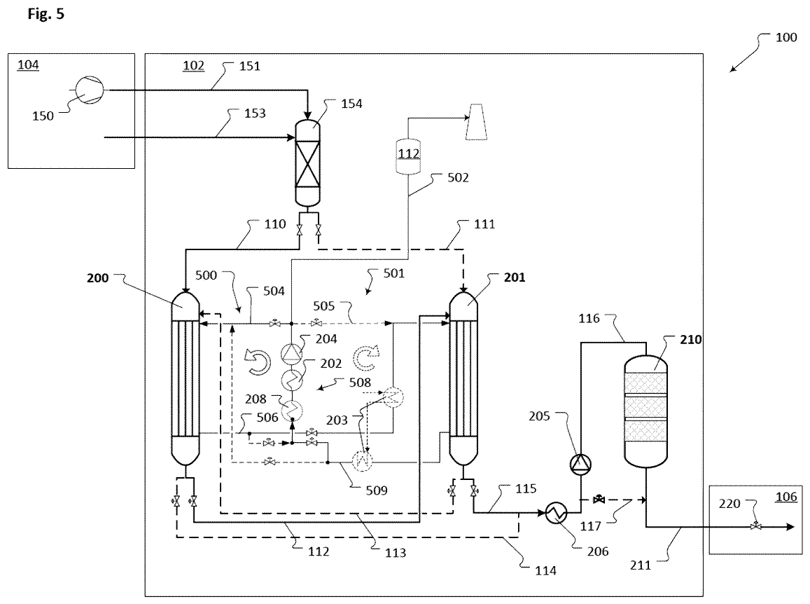

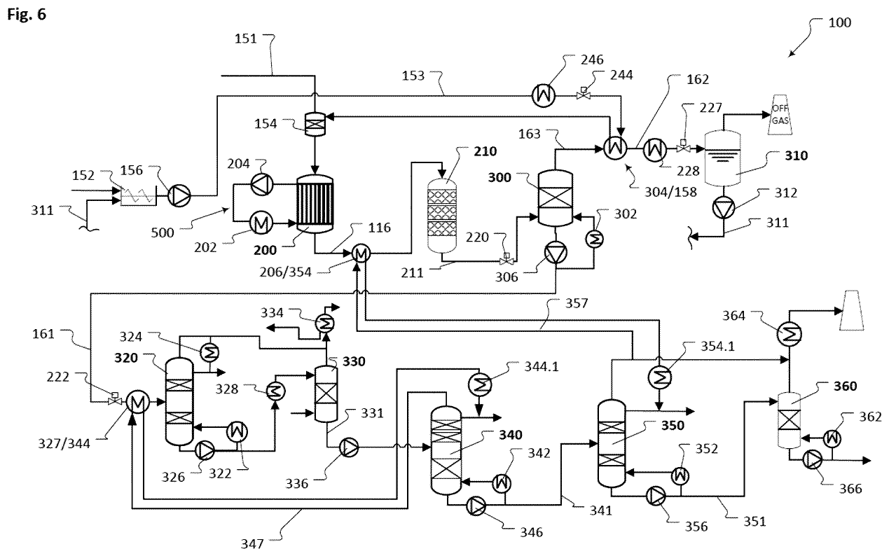

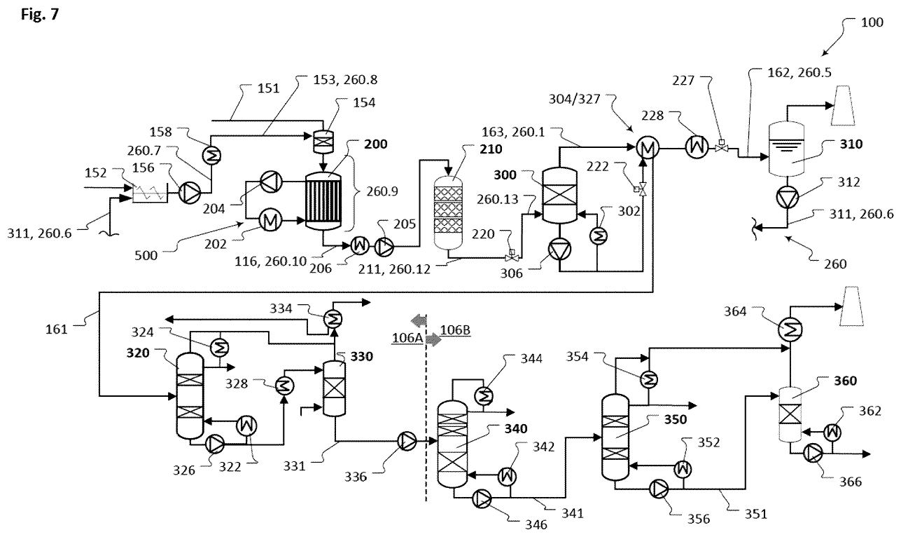

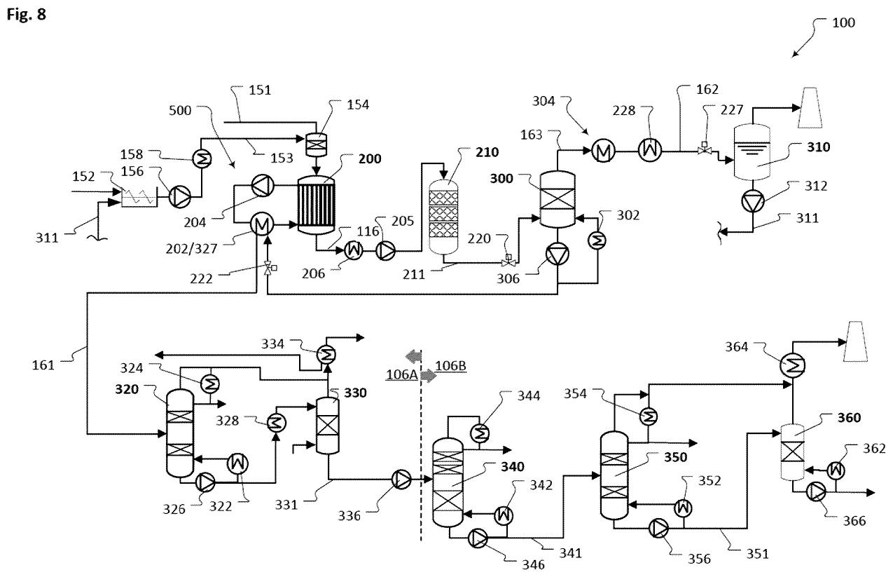

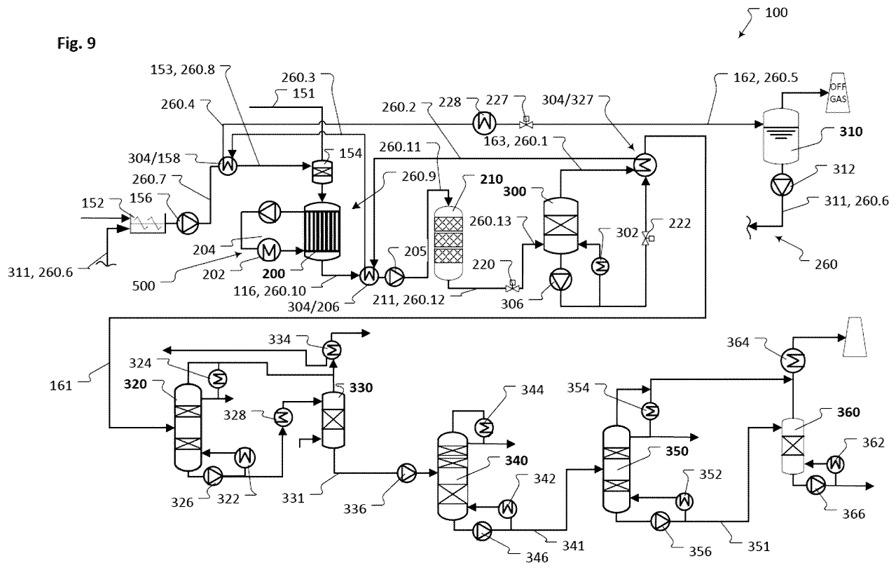

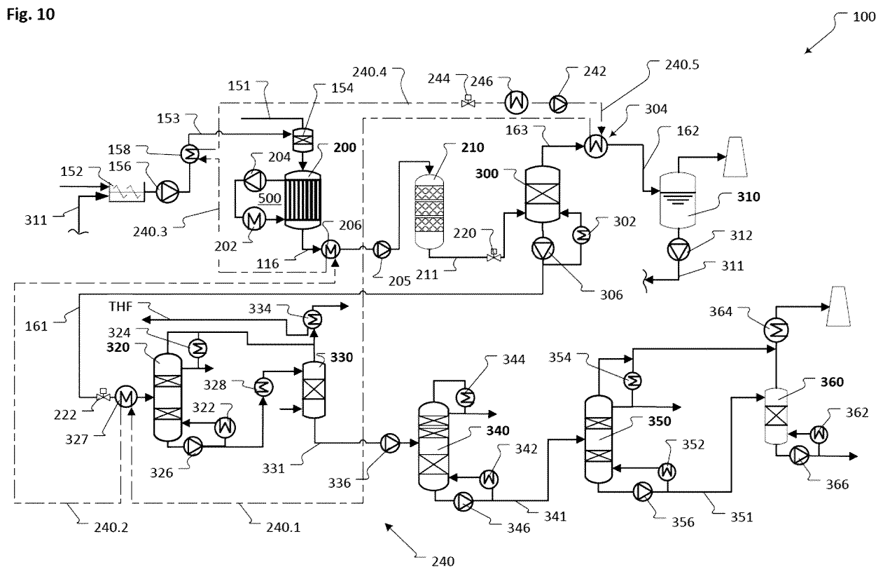

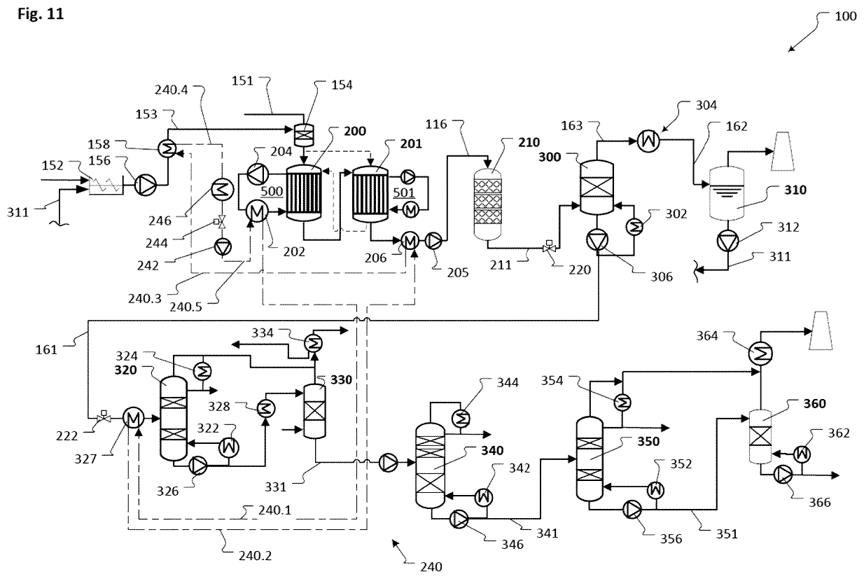

The solution according to the invention is described in detail hereinafter with reference to working examples. The figures show:
FIG. 1 a plant as a process flow diagram,
FIG. 2 a first embodiment of the plant with a first direct EC,
FIG. 3 a second embodiment of the plant with a further direct EC,
FIG. 4 a first embodiment of the reactor unit,
FIG. 5 a second embodiment of the reactor unit,
FIG. 6 (formerly FIG. 7) a third embodiment of the plant with a further direct EC as integrated material-based EC,
FIG. 7 (formerly FIG. 6) a fourth embodiment of the plant with a further direct EC as integrated material-based EC,
FIG. 8 (NEW) a fifth embodiment of the plant with a further direct EC as integrated material-based EC,
FIG. 9 a sixth embodiment of the plant with a further direct EC as integrated material-based EC with series interconnection and circulation,
FIG. 10 a seventh embodiment of the plant with a circuit for onward conduction of media and
FIG. 11 an eighth embodiment of the plant with an alternative circuit for onward conduction of media to FIG. 10.

FIG. 1 shows the plant 100 for continuous production of 4,4′-diaminodicyclohexylmethane (PACM) by catalytic hydrogenation of methylenedianiline (MDA; reactant1), especially 4,4′-diaminodiphenylmethane, with a hydrogen donor (reactant2), where the hydrogen donor is supplied in the form of gaseous hydrogen (H2). The plant 100 comprises a conditioning unit 104 for the reactants, a reactor unit 102 and a separation unit 106.

The conditioning unit 104 is framed by dashed lines and comprises (feed) conduits for the reactant1 and the hydrogen (reactant2), and also the solvent. In addition, the conditioning unit comprises a compressor unit 150, referred to hereinafter as compressor in the conduit 151 supplying the hydrogen, a mixer 152 in the conduit supplying the solvent and reactant1. Additionally disposed in the conduit 153 supplying the mixture of reactant1 and solvent are a pump 156 and a heat exchanger 158. The conduits 151, 152 open into a mixing vessel 154 disposed upstream of the main reactor 200. The mixing vessel 154 serves for intensive mixing of the reactants, and its outlet forms the inlet for the main reactor 200.

The reactor unit 102 is framed by dashed lines and comprises essentially the main reactor 200, a cooling circuit 500, a postreactor 210 and a heat exchanger 206 in the feed conduit to the postreactor 210. The cooling circuit 500 incorporates a pump 204 and a heat exchanger 202, where the circulating coolant in the main reactor 200 flows around the catalyst material-filled carrier elements. In the example shown, the flow direction toward the catalyst material-filled carrier elements is in cocurrent direction. The postreactor 210 is connected via the conduit 211 to the separation unit 106, i.e. the first separation stage thereof.

The heat exchanger 202 in the cooling circuit 500 is shown as an air-cooled heat exchanger 202, but may also be alternatively designed in order to control the temperature of the cooling medium in the cooling circuit 500 in indirect heat exchange, for example by means of a flowing cooling medium, such as an oil, water or a brine. The cooling circuit 500, in a plant variant which is not shown, also incorporates a heat exchanger, analogously to the heat exchangers 208, 209 in FIGS. 5 and 6. The latter is operated with a heating medium and serves, in the step of starting up the main reactor 200, to adjust the temperature of the main reactor 200 to about 80 to 100° C., ideally to a temperature of 85° C. to 95° C. In the plant and process example shown, in which one aim is to achieve a minimum trans/trans isomer ratio of about 17% to 23% by weight, the main reactor 200 filled with fresh or regenerated catalyst is preheated to a temperature of about 90° C. by means of the heat exchanger 208. The main reactor 200 is operated at a pressure of 87 to 88 bar.

The separation unit 106 is framed by dashed lines and comprises a plurality of separation apparatuses for separating the solvent, especially in a first separation stage, and the PACM product, especially in a second separation stage, from the rest of the reactant and by-products. The first separation stage (not displayed) comprises a separation tank 300 to which a bottoms circulation system is connected, incorporating a heat exchanger 302 and a pump 306. The top outlet of the separation tank 300 is connected to a heat exchanger 304, a condenser, downstream of which is provided a collecting vessel 310 for the solvent. By means of the heat exchanger 304 (condenser), about 80% of the solvent, THF in this case, is condensed out and could be collected or returned. In the flash stage, an energy requirement of about 1400 kW is required for the heat exchanger 302 in the bottoms circulation system of the separation tank 300.

The condenser 304 is shown as a heat exchanger in the design of an air-cooled apparatus, but may also alternatively be designed to at least partly condense and to cool the vaporous stream of matter at the outset in indirect heat exchange, for example by means of a flowing cooling medium such as cooling water or a medium suitable for thermal integration.

The amounts of energy specified herein are calculated for a plant output of the PACM product in the synthesis reaction mentioned of about 3.37 t/h, and a by-product output of about 0.4 t/h of HB and about 0.05 t/h of LB. The ratio of the mass flow rates of THF to MDA was 4.2 to 4.5. In addition, the first separation stage of the separation unit 106 for further removal of the solvent comprises a first separation column 320 and a second separation column 330, where the second separation column 330 takes the form of a stripping column. For this purpose, nitrogen (N2) is advantageously used as a stripping medium, which is passed through the column in countercurrent to the stream of matter. By means of the pump 306 disposed in the bottoms outlet of the separation tank 300, the solvent-depleted stream of matter can be diverted via the conduit 161 to the first separation column 320. A pressure control unit 222 is provided in the conduit 161 and is designed as a controllable valve in the example shown. The stream of matter is introduced into the first separation column 320 via a central inlet as shown. Additionally disposed upstream of the first separation column 320, as an optional embodiment, is a (forward) heat exchanger 327 (shown by dashed lines), which constitutes an option for heating of the first separation column 320.

In the first separation column 320, the solvent concentration is reduced from 30% by weight in the feed via the product-rich stream of matter from the conduit 161 to about 2% by weight of residual solvent.

This first separation column 320, equipped with (structured) packings, is connected to a bottoms circulation system incorporating a heat exchanger 322 and a pump 326. In addition, a tops circulation system is disposed at the column top of the first separation column 320, incorporating a heat exchanger 324 designed as a condenser. Two outlets for the solvent-rich stream of matter lead out of the tops circulation system, with one of the two outlets leading into the tops outlet of the downstream separation column 330 (stripping column).

In particular, the solvent separated off in the first separation stage is conductable via the conduit 311 into a collection tank (not shown) and/or the mixer 152 of the conditioning unit 104. In the conduit 211 leading from the postreactor 210 to the separation tank 300, a pressure control unit 220 is provided, designed in the present example as a controllable valve.

The product-rich stream of matter having a residual content of about 2% by weight of solvent (THF) is directed from the bottoms outlet of the first separation column 320 via the conduit 321 into the top of the second column 330, the stripping column. This conduit 321 incorporates a heat exchanger 328. The second column 330 has at least one internal packing, where a gas feed for an inert gas (stripping gas) in particular is disposed below the packing, such that the introduced product-rich stream of matter flows through the second column 330 in countercurrent principle to the stripping gas and is further depleted of solvent. In the plant example shown, nitrogen (N2) is used as stripping gas. The solvent-rich vapour is directed via the tops outlet into a condenser 334 and fed to the condenser 334 together with the vapour stream coming from the tops outlet of the first separation column 320 and introduced into the tops discharge of the second separation column 330. The solvent stream condensed out in the heat exchanger 334 is returned to the conditioning unit 104, wherein the uncondensable portion is led off from the heat exchanger 334 and, for example, fully thermally oxidized.

The product-rich stream of matter is fed from the bottoms outlet of the second separation column 330 to the third separation column 340 via the conduit 331, incorporating the pump 336. The second separation stage 106B of the separation unit 106 serves in particular to separate the LB and/or HB by-products from the PACM product.

The second separation stage (106B) comprises essentially three separation columns, where the third separation column 340, the first of the second separation stage, is supplied centrally with the stream of matter. The third separation column is connected to a bottoms circulation system incorporating a heat exchanger 342 and a pump 346. The product-rich stream of matter is directed via the conduit 341 from the bottoms outflow to the fourth separation column 350. In addition, the third separation column 340 is connected to a tops circulation system incorporating a heat exchanger 344. Condensed LB is led off as the first by-product from this tops circulation system.

The product-rich stream of matter is fed centrally to the fourth separation column 350 via the conduit 341. The fourth separation column 350 is connected to a bottoms circulation system incorporating a heat exchanger 352 and a pump 356. In addition, the fourth separation column 350 is connected to a tops circulation system incorporating a heat exchanger 354 in the form of a condenser. The product-rich stream of matter is discharged from the tops circulation system as condensate via the tops outlet. In the present case, according to the example shown, the uncondensed vapour stream and/or gas stream is introduced into the tops discharge of the downstream fifth separation column 360. Subsequently, product is condensed further and discharged via a further heat exchanger 364 (condenser). The stream of matter with a low level of product is discharged via the bottoms outlet of the fourth separation column 350 via the conduit 351 and introduced into the top portion of the fifth separation column 360. This stream of matter is highly enriched with HB, a second by-product. The fifth separation column 360 is connected to a bottoms circulation system incorporating a heat exchanger 362 and a pump 366. Furthermore, the separation column 360 has a tops outlet which leads to the aforementioned heat exchanger 364 in the form of a condenser. In this condenser 364, a further product-rich stream of matter is condensed out as condensate and discharged, with gaseous discharge of the uncondensable portion. In particular, the latter may subsequently be fully oxidized.

With regard to the stream of matter, the reactor unit 102 is operated at a first, high pressure level of about 60 to 120 bar, the first separation stage 106A of the separation unit 106 is operated at a second, low pressure level of 4 to 12 bar, and the first separation column 320 and the second separation column 330 are operated at a third, slightly elevated pressure level of 1.05 to 2.5 bar. In the example shown, the first pressure level is 80 to 90 bar, the second pressure level is 4.5 to 7.5 bar and the third pressure level is 1.1 to 1.2 bar.

With direct temperature control of the main reactor 200 via the cooling circuit 500 in series with the uncooled postreactor 210, surprisingly, a reduced energy requirement compared to the prior art and a simplified, more stable process regime has been demonstrated. Without being tied to any specific interpretation, this success is apparent in that the cooling circuit 500 only has to be specifically designed for the very vigorous initial reaction for dissipation of the exothermic heat of reaction, while the still significant postreaction has to be adjusted via the feed temperature by means of the upstream heat exchanger 206 alone. Because the reaction in the postreactor 210 is already highly attenuated, the stream of matter is only heated by about 5 to 15° C. and can be discharged into the separation tank 300 at this level without any problems.

All in all, the figures show internals such as packings, separation planes, support elements etc. in the apparatuses such as reactors, separation columns, vessels etc. by means of the corresponding symbols. These each indicate advantageous embodiments, with regard to the number, type and/or relative position to the respective feed conduit or discharge conduit. For example, the illustration of first separation column 320 thus indicates that, advantageously, the (feed) stream of matter is introduced via the conduit 161 in such a way that at least one (theoretical) separation plane is present in each case between the tops circulation system and the bottoms circulation system. The determination of the specific type and/or number of separation planes is known to the skilled person and can be varied or provided for in an appropriate manner.

FIG. 2 shows a first embodiment of a direct EC, where the plant 100 corresponds to that from FIG. 1. The dashed lines 230, 232, 234 show media conduits in which the heated medium from the heat exchanger 304 (condenser), disposed in the (tops) conduit 163 of the separation tank 300, is passed on directly as heating medium to further downstream heat exchangers 158, 206 and 327. The three media conduits 230, 232, 234 and the associated direct onward media conduits can be provided individually or collectively. The heat exchangers 158, 206, 327 are thus connected in parallel to one another, where all three heat exchangers serve as heat source for the condenser 304. The medium flowing to the condenser 304, water here, leaves the plant 100 downstream of the three heat exchangers 158, 206, 327.

FIG. 3 shows further embodiments for direct EC, as series media-based EC, where the plant 100 corresponds essentially to FIGS. 1 and 2. Analogously to FIG. 2, the heat exchanger 304 is connected as heat source to the heat exchangers 158 in the feed to the main reactor 200 and the (forward) heat exchanger 327 in the (feed) conduit 161 to the first separation column 320. The heat exchangers 158, 327 are connected in parallel to the condenser 304 in a series media-based EC and to one another. Furthermore, the (bottoms) heat exchanger 322 charged with superheated steam, for example, is connected in a media-conducting manner to the (forward) heat exchanger 206 of the postreactor 210 for the media conduit 238. The outflowing condensate or the water-vapour mixing phase leaves the (bottoms) heat exchanger 322 at a temperature of above 170° C. and serves as energy source for the (forward) heat exchanger 206 by direct onward conduction of energy/media, such that no additional energy consumption is required for the heat exchanger 206.

The media conduit 236 shows a media connection between the heat exchanger 354 of the fourth separation column 350 and a parallel second (bottoms) heat exchanger 302B to a steam-fed first heat exchanger 302A. Because of this redundancy with two bottoms heat exchangers 302A,B in the bottoms circuit of the separation tank 300, the energy requirement therein can be reduced by about 35%.

In the example shown in FIG. 3, the heat exchangers 304 (condensers) as heat source are coupled in a direct EC, i.e. series-connected, via media conduits to the heat exchangers 158, 206 and 327 operating as a heat sink. In this case, heat sinks incorporated are the heat exchanger 327 in the (feed) conduit 161 to the first separation column 320, the heat exchanger 206 in the feed to the postreactor 210 and the heat exchanger 158 into the (feed) conduit 153 upstream of the main reactor 200. This option of direct thermal coupling via media conduits is shown in the lower frame marked A in FIG. 3. This selective direct interconnection as direct EC by means of media conduits and distribution of the heat from the condenser 304, compared to the incorporation of the heat sinks into the distributor circuit 570 according to FIG. 2 for example, as described above, results in an increase in efficiency of 30% to 50%.

What is meant in the present context by “an increase in efficiency”, unless stated otherwise, is a lower energy consumption. The reference point will be apparent from the context and may be based on the plant, the plant section respectively described or the improved apparatus, for example via integration of two heat exchangers into a single one.

FIG. 4 shows an improved variant of the reactor unit 102. Two main reactors 200, 201 here are switchably connected to one another in series. Some of the valves/valve units that can be controlled by open- and/or closed-loop control are shown in FIG. 4; others can be provided if required by the person skilled in the art in order to ensure safe operation of the two main reactors 200, 201. The two main reactors 200, 201 are each incorporated into a cooling circuit 500, 501, with which the fixed bed reactor 200, 201 is kept in each case at a permissible, cooled reaction temperature. In the interconnection of the main reactors 200, 201 illustrated and shown with solid lines, the flow passes first through the first main reactor 200 via the conduit 110 coming from the mixing vessel 154 and the first part of the reaction takes place in this main reactor 200. Downstream via a lower outlet and the conduit 112, the mixture of matter is fed into the top of the second main reactor 201 and leaves it via a bottoms outlet and conduit 115 into the common conduit 116 as a feed into the common postreactor 210. The common conduit 116 incorporates a heat exchanger 206, by means of which the temperature level required for the postreactor 210 can be ensured. The sequence of the flow through the two main reactors can also be reversed, from the main reactor 201 to the main reactor 200. For this purpose, in an analogous manner, the flow to the main reactor 201 first passes through the conduit 111, with the conduit 110 to the top of the main reactor 200 closed. The mixture of matter leaves this fixed bed reactor via a bottoms outlet and the (intermediate) conduit 113 which is connected to the top of the main reactor 200, with the conduit 115 closed. Finally, the mixture of matter leaves the main reactor 200 via a bottoms outlet and conduit 114, which opens analogously into the common conduit 116 and thus leads to the postreactor 210. Each of the two cooling circuits 500, 501 has a conduit 502, 503 which branches off and is guided in each case through a container 212, 213, which serve as pressure equalization vessels, and further to a chimney and/or a complete oxidation unit. The conduits and valves/valve units are present and designed in such a way that each of the main reactors 200, 201 can be operated alone and the stream of matter can completely bypass the respectively other main reactor. The flow direction of the two cooling circuits 500, 501 is symbolized by an arrow, where the two cooling circuits 500, 501 are advantageously identical or substantially identical, since the two main reactors 200, 201 are operated alternately with respect to the flow direction of the reactant stream or the stream of matter as the first or second main reactor.

However, the cooling circuits 500, 501 are designed and controllable in such a way that, depending on the process-related requirements, such as, in particular, product management and/or product quality, autonomous cooling outputs or cooling functions are achieved in each case. This relates in particular to the volume flow per unit time of cooling medium and/or the temperature level or the permissible heating of the respective cooling medium. Furthermore, there is a direct option for thermal integration of the cooling circuits 500, 501 with other plant sections or heat exchangers, where the heat exchangers 202, 203 serve as heat source by receiving the heat of reaction, such that the total energy consumption of the plant or the process for producing PACM can be lowered.

FIG. 4 also shows an optional conduit 117 as a dashed line (bypass 2), by which the postreactor 210 can be bypassed if, for example, it has to be maintained and/or the catalyst charge has to be renewed. In this case, the temperature at least of the second main reactor in flow direction of the stream of matter is controlled such that complete reaction is ensured with the desired product quality, in particular the desired proportion of the respective isomers. The conduit branch of the conduit 117 may be provided upstream or downstream of heat exchanger 206 in flow direction, advantageously upstream of heat exchanger 206, in order also to be able to bypass it if necessary, and to be able to undertake necessary maintenance operations while the plant is running. Furthermore, there is a direct option for thermal integration of the cooling circuits 500, 501 with other plant sections or heat exchangers, where the heat exchangers 202, 203 serve as heat source by receiving the heat of reaction, such that the total energy consumption of the plant or the process for producing PACM can be lowered.

The main reactor 200 can be bypassed in operation of the main reactor 201 shown on the right in the picture via the conduits 111, 115 and 116. The main reactor 201 can be bypassed in operation of the main reactor 200 shown on the left in the picture via the conduits 110, 114 and 116, such that there is no flow through the (cross-) conduits 112, 113 between the two main reactors 200, 201 in each case. A pump 205 is disposed in the conduit 116.

The cooling circuits 500, 501 also (optionally) incorporate heat exchangers 208, 209 in the plant variant shown. These are operated with a heating medium, especially steam, and serve for (pre) heating the reaction temperature of the respective main reactors 200, 201 in the startup step of the main reactors 200. The level of this preheating is approximately 80 to 100° C., ideally 85° C. to 95° C. In the plant and process example shown, as already set out for FIG. 1, with a view to the aim of achieving a minimum trans/trans isomer ratio of about 17% to 23% by weight, it is advantageous when the newly catalyst-filled main reactor 200, 201 is preheated to a temperature of about 90° C. by means of the respective heat exchanger 208, 209, or the respective newly filled main reactor 200, 201 is correspondingly preheated.

The preheating to typically 85 to 95° C. makes it possible for the reaction in the case of the present catalyst to start immediately, without or substantially without recycling streams of the mixture of matter from the process until the desired reaction temperature has been attained.

The variant of the switchable main reactors 200, 201 as shown in FIG. 8 differs from that according to FIG. 7 in that the cooling circuit 500 of the first main reactor 200 is likewise connected in series with the cooling circuit of the second main reactor 201. In this case, only one (cooling) heat exchanger 202 and only one pump 204 is provided for the common cooling and the media circuit, and so the flow through the common (central) conduit branch 508 in both cooling circuits 500, 501 is generally constant and only in one direction, irrespective of the interconnection of the two main reactors 200, 201. Of course, the “central” conduit branch need not in fact be disposed between the two main reactors 200, 201. The variant is shown as solid lines, in which the first main reactor 200 (on the left) is first fed with the reactant mixture via the conduit 110 and also the introduction (of cooling media) downstream of the heat exchanger 202 and the pump 204 is first effected via this main reactor 200. The conduits that do not carry media in this circuit or conduits that carry the mixture of matter are shown by dashed lines. In these variants too, it is possible to completely bypass the respectively other main reactor with the mixture of matter and/or the (cooling) medium. For example, in the process of filling one of the two main reactors 200, 201, the respectively other main reactor can thus continue to be operated at maximum output. The bypass of the stream of matter or of the respective main reactor 200, 201 is analogous to FIG. 7.

In the example circuit shown, the flow passes through the central conduit branch 508 and the heat exchanger 202 and is directed via the conduit 504 in cocurrent into the media space of first main reactor 200, blocking conduit 505 that leads to the common conduit node coming from the second main reactor 201. The medium leaves the first main reactor 202 via the conduit 506 at a low-level outlet and is directed to a high-level inlet into the media space of the second reactor 201 (cross-conduit). The medium likewise flows through the media space of the second main reactor 201 in cocurrent and leaves it at a low-level outlet via the conduit 509, from which a branch again flows into the central conduit branch 508, and so flow through the circuit can continue. Similarly, the flow passes firstly via the conduit 505 through the main reactor 201 shown on the right when the conduit 504 is blocked. The medium then leaves the media space of the second main reactor 201 via the conduit 509 and is directed into the media space of the other main reactor 200 at a high-level inlet. The outlet of the media space at a low-level point leads into the conduit 506 and thence via a branch into the central conduit branch 508.

The particular advantage of the switchable main reactor 200, 201 is the possibility of operating the main reactor, through which the flow passes first, at a higher temperature because the catalyst is already partly exhausted and inactivated, essentially without adversely affecting (i.e. increasing) the trans/trans isomer ratio. At the same time, more significantly heated cooling medium is obtained in the respective (circulation) heat exchanger 202, 203 of the main reactor 200, 201 through which the flow passes first. Because of the higher temperature, this hotter heat exchange medium can be better used in the plant for integrated media-based EC and/or for customary heat exchange with a reactant stream of matter.

FIG. 5, analogously to FIG. 4, shows the optional conduit 117 as a dashed line (bypass 2), which allows the postreactor 210 to be bypassed, where the conduit 117 branches off downstream of the (forward) heat exchanger 206 of the postreactor 210.

FIG. 5 also shows a plant variant (dashed line) whereby higher control security or a higher degree of freedom of temperature management is obtained in the respective (cross-) conduit 504, 505 of the cooling circuits 500, 501 in that switchable and controllable heat exchangers 203 (feed coolers) are arranged as required. In the working example shown, the two heat exchangers 202 are connected in series, since, because of the respectively inactive (cross-) conduit, the conduit 505 in the example shown, no heating takes place at the site of installation for the heat exchanger 202. The cooling media conduit or the cooling media circuit of the heat exchangers 202 can be operated in an advantageous variant in a sidestream or via a secondary or auxiliary circuit via the pump 204.

The great advantage of this sidestream or secondary/auxiliary circuit of a coolant via the heat exchangers 203 is that this additional cooling output only needs to be called on as required and, with a low degree of complexity, a greater extent of closed-loop temperature control as required is possible in the respectively second of the two series-connected main reactors.

FIG. 6 shows a third embodiment of the plant with a further direct EC, as an improved variant of the plant variant shown in FIG. 1, for example. In the embodiment shown, a total of three heat exchangers are shown in integrated material-based EC. In order to establish reference with FIG. 1, the “/” notation is used throughout the figures for heat exchangers in integrated media-based EC or integrated material-based EC, for example condenser 304/158, which means that at least a portion of the heat transfer from formerly two heat exchangers is now effected in a single heat exchanger. The sequence of reference numerals here is of no further significance. In the example of FIG. 6, the complete elevation in temperature of the stream of matter in the (feed) conduit 153 is accomplished, while the cooling of the stream of matter in the conduit 163 may be incomplete, and so a heat exchanger 228 is provided as an at least optional postcondenser. This (on-demand) heat exchanger 228 downstream of the condenser 304 in the conduit 163, 162 may be of smaller design because the cooling function is comparatively minor. Ideally, the entirety of the heat exchange for both streams of matter takes place in a single heat exchanger in integrated material-based EC. In the present case, the conduit 163 is regularly intended to mean the entire conduit from the separation tank 300 to the collection vessel 310, and the conduit 162 to mean the conduit section between the condenser 304 and the collection vessel 310.

As a further integrated material-based EC, the stream of matter in the (tops) conduit 347 of the third separation column 340 is coupled with the stream of matter in the (feed) conduit 161 to the first separation column 320 in the heat exchanger 327/344, with provision, analogously to the above variant, of a very much smaller (tops) condenser 344.1 at the top of the third column 340 in order to ensure the requisite condensation of the vapour at the top of the separation column 340. The third integrated material-based EC is effected between the stream of matter in the (tops) conduit 357 of the fourth separation column 350 and the stream of matter in the (feed) conduit 116 to the postreactor 210. This third integrated material-based EC is effected in the heat exchanger 206/354. The change in temperature levels in the respective streams of matter is effected in the same way as described above. In an analogous manner to the second integrated material-based EC, a very much smaller (tops) condenser 354.1 is provided in the return leg at the top of the fourth column 340 in order to ensure the requisite condensation of the vapour at the top of the separation column 350 permanently or as required.

FIG. 7 shows a further direct EC of the plant 100, where the stream of matter of the (tops) conduit 163 of the separation tank 300 in the condenser 304, as an integrated material-based EC, is coupled in a heat-exchanging manner to the stream of matter from the (bottoms) outlet via the conduit 161 of the separation tank 300. In contrast to FIG. 1, a pressure control unit 222 is already disposed upstream of the condenser 304 in the conduit 161 in order to be able to control the required temperature gradient in the incoming stream of matter for the condenser 304 by expansion. The conduit 161, connected downstream of the condenser 304, is connected to the first separation column 320. The (tops) conduit 163, connected downstream of the condenser 304, is connected to the collection vessel 310. Analogously to FIG. 6, a pressure control unit 227 and/or a heat exchanger 228, which is switchable if required, may optionally be disposed in the (tops) conduit 163 in order to ensure complete condensation of the vapour phase in the conduit 162, 163 upstream of the collection vessel 310.

In this embodiment, a circuit 260 may be formed for the heat-exchanging stream of matter, comprising several conduit sections 260.n, and incorporating the mixer 152, main reactor 200, the postreactor 210, the separation tank 300 in the heat-exchanging material or reactant stream. Furthermore, as one of the conduit sections 260.n, the (tops) conduit 163, the (feed) conduit 162, the (return) conduit 311 and the (feed) conduit 116 are incorporated. The heat-exchanging stream of matter or reactant stream used is the solvent or the fraction of solvents in the respective stream.

This direct interconnection as EC of the streams of matter and distribution of the heat from the condenser 304, compared to the incorporation of the heat sinks into the distributor circuit 570 according to FIG. 2, as described above, results in an increase in efficiency of 30% to 50%.

FIG. 8, by contrast with FIG. 7 and the embodiment therein, shows a further EC in the plant 100, wherein the stream of matter in the (bottoms) conduit of the separation tank 300 and the (feed) conduit 161 to the first separation column 320 is coupled in a heat-exchanging manner to the (circulating) heat exchanger 202 in the coolant circulation system 500 of the reactor 200. By contrast with FIG. 1, and analogously to the examples in FIG. 6 and FIG. 7, a pressure control unit 222 is already disposed upstream of the (circulation) heat exchanger 202 in the conduit 161 in order to be able to control the required temperature gradient in the incoming stream of matter for the condenser 304 by expansion. The conduit 161, connected downstream of the (circulation) heat exchanger 202, is connected to the first separation column 320.

FIG. 9 shows a further direct EC of the plant 100, wherein, analogously to the variant according to FIG. 7, the stream of matter of the (tops) conduit 163 of the separation tank 300 in the condenser 304, as an integrated material-based EC, is coupled in a heat-exchanging manner to the stream of matter from the (bottoms) outlet via the conduit 161 of the separation tank 300. Upstream of the condenser 304, a pressure control unit 222 is disposed in the conduit 161 in order to be able to control the required temperature gradient in the incoming stream of matter for the condenser 304 by expansion. The conduit 161, connected downstream of the condenser 304, is connected to the first separation column 320. The (tops) conduit 163, connected downstream of the condenser 304, is connected to the collection vessel 310. A pressure control unit 227 and/or a heat exchanger 228, which is switchable if required, may optionally be disposed in the (tops) conduit 163 in order to ensure complete condensation of the vapour phase in the conduit 162, 163 upstream of the collection vessel 310. In contrast to FIG. 7, the (tops) conduit 163, however, is not connected directly to the collection vessel 310 downstream via the (feed) conduit 162. The heat exchanger 304/327 is connected in series as an integrated material-based EC to the heat exchangers 206, 304 that act as heat sinks and the (feed) conduit 116 to the postreactor 210, and is likewise connected downstream in series to the heat exchanger 158/304 as an integrated material-based EC, where the discharge conduit branch corresponds to the (feed) conduit 162 to the collection vessel 310. This energy interconnection for onward conduction and distribution of the thermal energy is also shown in the variant as heat exchange circuit 260, where, for easier understanding, the conduit sections 260.n of the circuit 260 are numbered sequentially as 260.1 to 260.13. The return conduit 311 corresponds to the conduit branch 260.6 and conducts the heat-conducting solvent, in this case for example THF, from the first separation stage 106A back into the conditioning unit 104. If necessary, advantageous collection or storage tanks of the conditioning unit 104 are optionally incorporable into the heat exchange circuit 260.

In this embodiment, a circuit 260 may be formed for the heat-exchanging stream of matter, comprising several conduit sections 260.n, and incorporating the mixer 152, main reactor 200, the postreactor 210, the separation tank 300 in the heat-exchanging material or reactant stream. Furthermore, as one of the conduit sections 260.n, the (tops) conduit 163, the (feed) conduit 162, the (return) conduit 311 and the (feed) conduit 116 are incorporated. The heat-exchanging stream of matter or reactant stream used is the solvent or the fraction of solvents in the respective stream.

Incorporation and integrated material-based EC of the (forward) heat exchanger 327 upstream of the first separation column 320 has been found to be particularly surprising and advantageous because this separation column can be operated at a very low pressure level of about 1.1 bar to 2 bar in the conduit 161 according to the pressure control unit 222 and hence the temperature level of the stream of matter that flows to the heat exchanger 327 is very low at about 90° C. This means that it is possible to absorb relatively large amounts of heat in energy coupling and to economize on cooling output; in addition, the energy demand of the bottoms circuit of the first separation column 320 is almost linear. In the case of integrated material-based EC, it is possible in this way, based on the overall energy demand of the second separation column 320, to save, for example,

| | |
|---|---|
| in the heat exchanger 327/344 | about 31%, |
| in the heat exchanger 327/354 | about 62%, |
| in the condenser 327/304 | about 21% and/or |
| in the (circulation) heat exchanger 327/202 | about 12%. |

FIG. 10 shows an embodiment or variants formed analogously to the variants of FIGS. 2, 3. In this case, a heat circuit 240 is designed as a series interconnection of heat exchangers for integrated media-based EC. In this case, the condenser 304 serves as central heat source, and the (forward) heat exchanger 327 in the (feed) conduit 311, the (feed) heat exchanger 206 in the (feed) conduit 116 and the heat exchanger 158 in the (feed) conduit 153 are incorporated via n conduit sections 240.n. For example, the heat exchange medium used is water which is circulated in the pipe sections 240.n. It is a feature of the conduit sections of a heat exchange circuit 240, but also of the aforementioned heat exchange circuits 250, 260 that the temperature level and/or pressure level is different in the nth conduit section compared to the conduit section n−1 (upstream) and/or conduit section n+1 (downstream). In the examples and variants shown here, the conduit sections shown and/or displayed are illustrated by way of example, and so others may be provided on the basis of the respective requirements, especially when further heat exchangers are incorporated analogously in the heat exchange circuit 240, 250, 260 as a heat sink and/or further heat exchangers as a heat source.

In the conduit sections 240.4, 240.5, a pressure control unit 244 and a heat exchanger 246 operating as a heat sink are arranged upstream of the condenser 304 in the variant of the plant 100 shown, in order to ensure the required cooling/condensation output of the condenser 304.

The working example of FIG. 11, finally, corresponds in its basic design to that of FIG. 10, except that it is not the condenser 304 downstream of the separation tank 310 that serves as the central heat source of the heat exchange circuit 240, but the (circulation) heat exchanger 202 of the reactor 200. In an analogous manner, a pressure control unit 244 and a heat exchanger 246 working as a (cooling) heat sink are disposed in the transition from the conduit sections 240.4 to the last conduit section 240.5 upstream of the (circulation) heat exchanger 202, in order to ensure the required cooling capacity of the (circulation) heat exchanger 202 and thus to be able to optimally control the reactor 200.

A significant advantage of the (distributor) circuit 240 can be considered to be that a central steam circuit has been created and hence steam can be generated centrally, for example in a condenser 304, and distributed to all heat exchangers that operate as consumers (heat sinks), instead of directly interconnecting heat sources and heat sinks. Furthermore, the distributor circuit as a central steam circuit, if required (for example when starting up the plant), can be fed at least temporarily by means of an alternative heat or steam source.

The (forward) heat exchanger 206 is described primarily in the present context and is in some cases shown in an EC in which it operates as a heat exchanger 206 primarily as a heat sink, meaning that the stream of matter conducted therein is heated. Because of the dependent mode of operation of the postreactor 210 with adaptation to the main reactor 200, 201, cooling of the stream of matter in the (feed) conduit 116 upstream of the postreactor 210 may be required at least temporarily, in particular permanently, because about 10% to 20% of the conversion occurs in the (adiabatic) postreactor, such that the stream of matter is heated up to about 140° C., measured in the outlet. Thus, independently of the embodiments and variants of the plants and of the process that are described herein, it is possible to provide an adapted EC for cooling (heat exchanger 206 operates as a heat source). Alternatively or additionally, supplementary cooling may be provided via a modified or additional EC.

Overall, a multitude of customary open-loop and closed-loop control elements that are known to the person skilled in the art and are necessary or advisable for an advantageous process regime are not shown, such as sensors (flow, temperature, pressure etc.), displays, setting and control elements (especially valves, further pumps, compressors), collecting vessels etc., and should be added if required. In particular, when “a” pump or “a” compressor is mentioned, this also means customary redundancies from at least two parallel aggregates, especially of two parallel pumps or two parallel compressors. In an analogous manner, “a heat exchanger” should not be understood to be limiting and, with the respective local heat exchange function, also means arrangements of heat exchangers that are connected in series or in parallel, including redundant heat exchangers, which in this context does not mean interconnections with at least one further heat exchanger and a locally different heat exchange function.

All heat exchanges and condensers are fundamentally designed such that indirect heat transfer occurs and there is no physical mixing of reactants, product, by-products and/or solvent with the (heating/cooling) medium, such as gas, steam, water, oil, brine etc., unless stated otherwise.

Even if components such as valves, pressure control unit, isolators etc. are shown individually or separately in the present context for simplified description and to some degree were not mentioned individually, this should not be read in a limiting manner; instead, the person skilled in the art is able to combine two or more of these components in a valve unit or control unit as required or provide multiway valves instead.

Owing to the consideration of media streams, reactant streams and streams of matter to the EC, the terms “upstream” or “downstream” are interpretable only from the respective context.

The terms “bottoms outflow”, “bottoms output”, “bottoms outlet” or “bottoms discharge” are to some degree used synonymously, as are analogously the terms “tops outflow, output, outlet or discharge”.

Furthermore, the expression “heat exchanger operated as a condenser” should be interpreted broadly, and also means incomplete condensation or cooling of the supplied stream of matter, and so a “heat exchanger operated as a condenser” is also synonymously named “condenser” in some cases.

The term “bottoms pump” means a pump incorporated into a bottoms circulation system of an apparatus (separation column, vessel, etc.) and/or a pump downstream of the bottoms outlet of an apparatus, which is intended to convey liquid stream of matter.

Beyond the examples shown herein, it has been found to be surprisingly advantageous and effective overall to provide, downstream of the expansion unit, a forward heat exchanger in the (feed) conduit to the first separation column of the first separation stage, and advantageously to provide this in an EC, especially in an integrated EC.

For all embodiments and variants of the plant and of the process, it may generally be the case that the second separation column 340 and the third separation column 350 are executed as a single column, in particular as a dividing wall column (not shown). In this case, the separation functions of the second separation column 340 and of the third separation column 350 can advantageously be performed at least partly, ideally completely, by means of the dividing wall column (not shown), as known, for example, from documents EP 012 62 88 B1 or EP 012 23 67 A2.

Overall, a large, energy benefit can be achieved with the plant according to the invention and the process, wherein a significant reduction in the externally supplied energy flows was enabled, in particular saving of large amounts of (external) heating steam.

1. A plant for catalytic hydrogenation of methylenedianiline (MDA; reactant1) with a hydrogen donor (reactant2), the plant comprising:
a conditioning unit for the reactants,
a reactor unit, and
a separation unit,
wherein the conditioning unit comprises (feed) conduits for reactant 1, reactant 2 and at least one solvent, at least one heat exchanger in at least one (feed) conduit, at least one mixer for mixing the reactants and/or at least one reactant with at least one solvent;
wherein the reactor unit comprises at least one fixed bed reactor as main reactor with an immobile catalyst packing, wherein the at least one main reactor comprises a first flow pathway for the mixture of matter through the immobile catalyst packing and a further flow pathway, and
wherein the further flow pathway incorporates a heat exchanger for influencing a temperature level in the first flow pathway;
wherein the separation unit comprises at least a first separation stage comprising at least one apparatus for removing the at least one solvent, where the at least one apparatus is connected downstream to at least one condenser via at least one (tops) conduit, and
a second separation stage comprising at least one apparatus for separation of at least one reactant and/or at least one by-product from the product, where the at least one apparatus is connected downstream to at least one condenser via at least one (tops) conduit,
wherein
the further flow pathway is a closed media circulation system for a heat transfer medium, at least a section of which runs outside the immobile catalyst packing of the at least one main reactor for indirect heat transfer, where the closed media circulation system incorporates at least one heat exchanger.

2. The plant according to claim 1, wherein the reactor unit comprises at least one postreactor series-connected downstream of the at least one main reactor, where the at least one main reactor and the at least one postreactor are incorporated via a conduit.

3. The plant according to claim 1, wherein at least one energy coupling (EC) is provided, incorporating at least one of the following heat exchangers:
the at least one condenser of the first separation stage,
the at least one condenser of the second separation stage,
the at least one heat exchanger of the closed media circulation system,
and where the EC means:
i) integrated material-based energy coupling (integrated material-based EC) of at least two streams of matter in a heat exchanger in indirect heat exchange;
ii) integrated media-based energy coupling (integrated media-based EC) of at least two heat exchange media in a heat exchanger in indirect heat exchange;
iii) serial interconnection of two heat exchangers in media-based energy coupling (serial media-based EC), where
in serial interconnection of the first heat exchanger in integrated media-based EC, a (heat exchange) medium is passed onward to a second downstream heat exchanger for further integrated media-based EC.

4. The plant according to claim 3, the plant comprising:
iv) at least one serial interconnection of two heat exchangers to the integrated material-based EC (“serial integrated material-based EC” for short), where a stream of matter from the first heat exchanger in integrated material-based EC is passed onward in serial interconnection to the second downstream heat exchanger for further integrated material-based EC.

5. The plant according to claim 3, wherein at least one serial media-based energy coupling according to iii) or a serial integrated media-based EC according to iv) is designed as a circuit.

6. The plant according to claim 3, wherein at least one of the following conduits in the at least one condenser is incorporated in the (tops) conduit of a separation tank as heat source for integrated material-based EC as follows:
i) the (feed) conduit to the main reactor,
ii) the (feed) conduit to the postreactor,
iii) the (feed) conduit to a first separation column, where a pressure control unit is disposed in the (feed) conduit upstream of the at least one condenser,
iv) at least one further heat exchanger for integrated material-based EC is incorporated downstream of an incorporation of a conduit according to i), ii) or iii) in the at least one condenser and upstream of a collection vessel.

7. The plant according to claim 6, comprising:
a serial media-based EC as circuit with a plurality of conduit sections, incorporating at least the following apparatuses and/or conduits:
the collection vessel downstream of the separation tank,
the (tops) conduit as a conduit section, and
the (feed) conduit to the separation tank as a further conduit section.

8. The plant according to claim 7, wherein the at least one condenser according to alternative iii) is in integrated material-based EC and is incorporated into the circuit.

9. The plant according to claim 8, wherein
the closed media circulation system of the main reactor as heat source is incorporated in the heat exchanger with the (feed) conduit to the first separation column to form the EC, where a pressure control unit is disposed in the (bottoms) conduit upstream of the heat exchanger.

10. The plant according to claim 9, wherein
at least one of the following conduits is incorporated in a condenser of a separation column of the second separation stage in the respective (tops) conduit as heat source for integrated material-based EC as follows:
i) the (feed) conduit to the main reactor,
ii) the (feed) conduit to the postreactor,
iii) the (feed) conduit to the first separation column of the first separation stage, where a pressure control unit is disposed in the (feed) conduit upstream of the at least one condenser of the respective separation column.

11. The plant according to claim 10, wherein at least one of the following direct media conduits is provided between heat exchangers for direct energy coupling (direct EC):
i) condenser of the separation unit as heat source for at least one heat exchanger of the reactor unit, the conditioning unit and/or the separation unit,
ii) heat exchanger of the closed media circulation system to the main reactor of the reactor unit as heat source for at least one heat exchanger of the reactor unit, the conditioning unit and/or the separation unit and/or
iii) heat exchangers among the reactor unit, the conditioning unit and/or the separation unit.

12. The plant according to claim 11, wherein the media conduit for direct EC:
i) leads from the at least one condenser of the separation tank of the separation unit as heat source to at least one of the following heat exchangers of the reactor unit, the conditioning unit or separation unit:
heat exchanger in a (feed) conduit to the main reactor and/or an apparatus disposed upstream of the main reactor,
heat exchanger in the (feed) conduit to the postreactor and/or
heat exchanger in the (feed) conduit to the first separation column of the separation unit, and where at least two of the heat exchangers are configured to be connected in parallel to one another;
ii) leads from a (cooling) heat exchanger of the reactor as heat source to at least one of the following heat exchangers:
heat exchanger in a (feed) conduit to the main reactor and/or an apparatus disposed upstream of the main reactor,
heat exchanger in the (feed) conduit to the first separation column of the separation unit, and where at least two of the heat exchangers are configured to be connected in parallel to one another; and/or
iii) leads from one of the (cooling) heat exchangers in the tops circulation system of a separation column of the separation unit to at least one of the downstream (heating) heat exchangers in the feed or bottoms circulation system of an apparatus of the separation unit.

13. The plant according to claim 12, wherein
the separation unit in the first separation stage, in the (feed) conduit to the separation tank, comprises at least one pressure control unit with a condensation unit for the at least one solvent, where a (return) conduit for the at least one solvent leads from the at least one condensation unit of the first separation stage to the conditioning unit.

14. The plant according to claim 13, wherein the reactor unit comprises a further main reactor in form of a fixed bed reactor comprising
a first flow pathway for the reaction mixture and
a further flow pathway as media circulation system for a heat exchange medium,
and where, by a valve unit provided in the (feed) conduit upstream of the two main reactors, a volume flow rate of the reactant mixture is divisible, conductable and/or completely switchable between the first main reactor and the further main reactor, and wherein each main reactor is connected to one heat exchanger or a common heat exchanger.

15. The plant according to claim 14, wherein at least one heat exchange circuit is included in form of a series integrated material-based EC having a plurality of conduit sections incorporating at least the following apparatuses and heat exchangers and connected via at least one of the conduit sections,
i) as heat exchanger and as heat source for the heat exchange circuit
the (circulating) heat exchanger of the main reactor, or
the (tops) heat exchanger in the (tops) conduit of the separation tank, each in integrated material-based EC with at least one of the following conduits with the reactant or stream of matter conducted therein as heat sink for the heat exchange circuit:
a) the (feed) conduit to the first separation column downstream of the pressure control unit,
b) the (feed) conduit to the postreactor,
c) the (feed) conduit upstream of the reactor, or the (return) conduit to the conditioning unit upstream of the mixer;
ii) as apparatus, at least the main reactor, at least one separation tank, at least one collection vessel of the first separation stage, and wherein
iii) the following conduits are integrated as conduit sections of the heat exchange circuit:
a) a (the first) conduit section corresponding to the (tops) conduit of the at least one separation tank and/or
b) a (last) conduit section corresponding to the (feed) conduit to the at least one collection vessel.

16. A process for catalytic hydrogenation of methylenedianiline (MDA; reactant1) with a hydrogen donor (reactant2), the process comprising:
effecting the catalytic hydrogenation by an industrial plant,
wherein
the industrial plant is designed according to claim 15, where the main reactor is operated at a temperature in a range of 80° C. to 150° C., and wherein, by EC:
i) energy is transferred from the at least one condenser of the separation unit as heat source for at least one heat exchanger of the reactor unit, the conditioning unit and/or the separation unit by integrated energy coupling (integrated EC) or direct energy coupling (direct EC) in a range of 5% to 100% of the available heat;
ii) heat exchanger of the media circulation system for the main reactor of the reactor unit as heat source for at least one heat exchanger of the reactor unit, the conditioning unit and/or the separation unit transfers energy by integrated EC or direct EC in a range of 5% to 30% of the available amount of heat; and/or
iii) heat exchangers of the separation unit exchange energy by integrated EC or direct EC in the range of 5% to 100% of the available amount of heat.

17. The process according to claim 16, wherein a temperature of the reactant stream at an inlet of the main reactor is 90 to 140° C.

18. The process according to claim 16, wherein the process is implemented continuously and catalytically for production of methylenebis(cyclohexylamine).

19. The process according to claim 16, comprising:
operating the at least one main reactor at a pressure in a range from 60 bar to 120 bar.

20. The process according to claim 16, wherein, in addition to the at least one main reactor, at least one postreactor comprising an immobile catalyst is also included, where the at least one main reactor and the at least one postreactor are operated at the same or essentially at the same pressure.

21. The process according to claim 20, wherein, from time t₀, the start of the process after renewal or regeneration of the immobile catalyst, to time t₄, the end of the process determined by renewal or regeneration of the immobile catalyst, the operating temperature of the main reactor is increased and the temperature in the stream of matter in the inlet (feed) to the postreactor is maintained or lowered, where the increasing or lowering of the temperature is linear and/or stepwise.

22. The process according to claim 16, wherein the MDA (reactant1) comprises a mixture of the following monomers: 4,4′ MDA, 2,4′ MDA and 2,2′ MDA, where a proportion of 4,4′ MDA is advantageously in a range from 75 to 98 mol %.
